(19) United States
(12) Patent Application Publication
(10) Pub. No.: US 2024/0292844 A1
(43) Pub. Date: Sep. 5, 2024

(54) Antimicrobial and Biological Active Polymer Composites and Related Methods, Materials, Coatings and Devices

(71) Applicants: David John VACHON, Spokane, WA (US); Mark Robert Vachon, Spokane, WA (US); IASIS MOLECULAR SCIENCES, INC., Spokane, WA (US)

(72) Inventors: David John VACHON, Spokane, WA (US); Mark Robert VACHON, Spokane, WA (US)

(21) Appl. No.: 18/290,139

(22) PCT Filed: May 7, 2022
(86) PCT No.: PCT/US22/28233

§ 371 Date: Nov. 9, 2023

Related U.S. Application Data
(60) Provisional application No. 63/186,173, filed on May 9, 2021.

(51) Int. Cl. A01N 59/20; A01P 1/00; A01P 3/00; A61L 15/18; B01J 39/09; B01J 39/20; C09D 5/14

(52) U.S. Cl. CPC A01N 59/20; A01P 1/00; A01P 3/00; A61L 15/18; B01J 39/09; B01J 39/20; C09D 5/14

(57) ABSTRACT

Antimicrobial ion exchange polymer salts are made by exchanging biologically active ionic agents onto organic ion exchange polymers. The activated polymers are useful in a wide range of antimicrobial coatings, materials, devices, agricultural treatments, and other applications, and are stable to thermal degradation and decomposition. The activated ion exchange polymer salts may be water-soluble or insoluble in water. Particle size distribution of the activated ion exchange polymer salts may be reduced and the resulting powders processed into polymers or polymer precursors using novel methods to produce stable, biologically active polymer composites, including antimicrobial polymer composites and coatings that are effective against bacteria, fungi and/or viruses. Antimicrobial polymer composites and coatings described here are potent against normal and drug resistant microbes (e.g., MRSA, and Candida auris) and have been proven effective against problematic human pathogenic viruses, including influenza viruses (e.g., H1N1) and noroviruses.

### RELATED APPLICATIONS

This application claims priority to U.S. Provisional patent application Ser. No. 63/186,173, filed 9 May 2021, and U.S. Provisional patent application Ser. No. 63/316,398, filed 3 Mar. 2022, each incorporated herein by reference in their entirety for all purposes.

### TECHNICAL FIELD

The invention relates to antimicrobial active materials, coatings, products and devices.

### BACKGROUND

Increased human population and global travel have facilitated pathogen transmission, rendering it more difficult to control disease spread. Before the late 1980s, the Centers for Disease Control and the American Hospital Association considered patients to be the principal vector for nosocomial pathogen transmission (infections occurring in hospitals or other healthcare facilities), largely ignoring risks arising from microbial contamination of inanimate surfaces (Cozad, A., and R. D. Jones. 2003). It is now well established that fomites (objects and surfaces vulnerable to contamination by pathogenic microorganisms) play a key role in spreading many infections in hospitals and other environments. (Aitken, C., and D. J. Jeffries. 2001, Barker, J., D. Stevens, and S. F. Bloomfield. 2001).

Fomites can be directly contaminated by contact with body secretions, aerosolized fluids, soiled hands, or other direct contact. Fomites can also be contaminated by airborne bacteria, fungi and viruses, which can be aerosolized by talking, sneezing, coughing or vomiting by infected subjects, or by suspension and settling of airborne microorganisms from other contaminated fomites. Transfer and transmission of infectious microbial pathogens between fomites, and from fomites to living hosts, are now well documented as very substantial infection pathways (England, B. L. 1982: Haas et al., 1999; Goldmann, D. A. 2000; Sattar, S. A. 2001; Reynolds et al., 2005).

As rates of fomite-mediated disease transmission have increased in hospitals, hospices, long-term care facilities, dental offices and other health care facilities, so too have these rates increased in other public environments, including daycare facilities, schools, gyms, jails and prisons, arenas and auditoriums, cruise ships, public transportation ports and vehicles, and at many other public sites and facilities.

In view of the foregoing, there is a long unmet need in the art to develop materials for use in healthcare facilities, food processing facilities, and other public environments that will minimize or prevent colonization and survival of bacteria, fungi, and viral pathogens on fomites located in these facilities and environments, to prevent transfer and transmission of pathogenic microorganisms between fomites and from fomites to susceptible human and veterinary subjects.

Notably, many pathogenic bacteria, fungi and viruses have exhibited resistance to conventional antimicrobial, antifungal or antiviral drugs, hence these pathogens are referred to as Drug Resistant (DR), Antibiotic Resistant (ABR); Antimicrobial Resistant (AMR), or MultiDrug Resistant (MDR) pathogens. There is accordingly a related need for materials, coatings, products and devices that will protect against colonization and survival of drug resistant bacterial, fungal, and viral pathogens on fomites in public environments, to prevent transfer and transmission of pathogenic microorganisms that exhibit drug resistance or other forms of resistance, between these surfaces and to susceptible living subjects.

Additional objects that remain unsatisfied in the art include development of contamination-resistant, contamination-preventive and infection-preventive materials that can be utilized across a wide variety of materials, devices, equipment, contact and touch surfaces, and public environments.

A related need exists for versatile polymer materials having a broad range of intrinsic surface biologically active properties, where the materials can incorporate a large diversity of antimicrobial surface-active agents and can be incorporated into a diverse array of compositions, coatings, materials and devices for broad adaptation and use within clinical, industrial, and public settings.

### BRIEF DESCRIPTION OF THE DRAWINGS

FIG. 1 is a graphic illustration of a filtration column (filter pack) comprising antimicrobial polymer composites of the invention for removal of microorganisms from water sources.

FIG. 2 depicts a clinical environment with numerous potential sources of microbial (bacterial, fungal and viral) contamination surrounding a patient, which sources (generally “fomites”) can be effectively, antimicrobially surface activated using antimicrobial polymer composites of the invention.

FIG. 3 illustrates a critical care environment, wherein an array of antimicrobially constructed and/or coated devices of the invention can be employed for patient care, for example to care for severely ill covid-19 patients. These patients depend on various medical devices, including tubular devices, for life support, each of which can be fabricated using the antimicrobial composites described herein.

### SUMMARY OF EXEMPLARY EMBODIMENTS OF THE INVENTION

The invention fulfills the foregoing needs and satisfies additional objects and advantages, by providing novel, insoluble and soluble organic and organometallic polymer materials that are biologically active, for example antimicrobially active. In exemplary embodiments the invention provides polymer materials incorporating ionic biologically active agents, for example ionic antimicrobial agents (e.g., ionic forms of metals and organic molecules to include oligodynamic metals, antibiotic agents, antiseptic agents, antifungal agents, and antiviral agents, etc.).

The incorporation or association of biologically active ionic agents with novel polymeric materials and coatings of the invention is achieved by combining one or more ionic biologically active agents with an organic-based, ion exchange polymer (weak or strong exchangers) in a salt or acid form, such as a functionalized organic-based ion exchange resin material. Organic and organometallic ion exchange resin materials useful within the invention may be carbon-based, making these materials fundamentally distinct from inorganic ion exchange materials such as mineral-based zeolites. Zeolites are known to lack compatibility with organic polymer materials to allow the facile creation of composite materials, very likely resulting from weak Van de Waals interactions between the mineral structure and the polymer matrix into which it can combined by casting, molding or extruding.

In exemplary embodiments of the invention, an organic porous ion exchange resin material is combined with a cationic or anionic biologically active agent (e.g., a cationic antibiotic, oligodynamic metal, or anionic anticoagulant) in an aqueous medium under conditions that mediate substitution of the ionic active agent onto the organic resin (e.g., by salt exchange or protonation), typically involving displacement of a similarly charged, anionic or cationic counter-ion originally bound to or associated with (i.e., ionically bound, electrostatically surface-associated, or adsorbed) the organic resin or non-resin polymer, to form a substituted, biologically active polymer salt. Organic ion exchange resin materials are typically insoluble, crosslinked polymers comprising functional groups that possess binding affinities for charged (cation or anion) compounds. The functionalities include sulfonate, carboxylate, phosphate, and ammonium for example. Under the proper conditions the charged compounds can be bound to the resin in high, measurable concentrations defined herein as “exchange capacity”.

In more detailed embodiments of the invention, a “biologically active” organic ion exchange polymer salt is constructed by ionically modifying the polymer salt to carry an ionic biologically active agent, for example, ionic silver (Ag+) substituted for a like-charged counter-ion originally bound on the organic resin, for example ionic sodium (Na+) or the addition of Ag+ by the replacement of H+. The resulting activated ion exchange polymer salt material is processed using novel materials and methods. In certain embodiments, larger, biologically active particles of the polymer salt are processed using a novel size reducing milling technology to generate a fine particulate, activated organic/organometallic ion exchange resin product, milled to a high degree of particle size uniformity.

The biologically active polymer salts of the invention are useful alone and in a diverse array of antimicrobial and other biologically active polymer “composite” materials. Within certain embodiments, a biologically active fine particulate ion exchange polymer salt material is combined with a thermoset or thermoplastic or photocuring polymers, solvent soluble or miscible polymers, or with water soluble or water miscible polymers to yield mixtures capable of forming solid activated polymer composite materials and surfaces.

Dry bead milling is especially useful for resins incorporating multivalent cations. Examples include Cu(II), Zn(II), Fe(II), Ga(II), Ce(III), Bi(III) and Ti(II) salts of weak cation exchange resins and strong cation exchange resins (WCERs and SCERs) although dry bead milling can be successful for monocation-modified resins as well.

Exemplary compositions of the invention are alternatively referred to herein in abbreviated form, by adding the incorporated ion at the beginning of the acronym followed by the resin type, e.g., Ag-SCER, Silver strong cation exchange resin), Cu(II)-WCER (Copper (II) or cupric ion weak cation exchange resin), BA (benzalkonium etc.,) such as BA-SCER. Other examples include Iron (II)-SCER, Fe(II)-SCER.

The methods, materials and composites of the invention can employ or integrate a large diversity of antimicrobial agents and activities. In additional embodiments, these methods, materials and composites can incorporate a host of other types of biologically active, ionic or ionizable agents, including a diverse array of clinically useful and therapeutic agents. In certain embodiments of the invention, fine particulate active organic resin materials are incorporated into solid polymers to create solid polymer composites, and these materials provide an astounding array of useful constructs, textiles, objects, devices, coatings, laminates and the like for use in healthcare, institutional, personal, medical, environmental, laboratory and other settings. In exemplary applications, the materials and constructs of the invention are useful in medical, dental, and veterinary facilities, tools, materials, implants, devices and equipment. In other exemplary embodiments the biologically active organic resin materials are incorporated into solid polymers to generate polymer composites and the composites are pelletized for uses that can include molding, extrusion, and other processing methods. Other uses and constructions of the materials and methods herein are described for consumer products, textiles, apparel, athletic equipment and accessories, sports therapy and gymnasium facilities and equipment, lavatory and food service materials, food packaging, food and beverage delivery, fluid transfer tubing, food processing equipment, medical equipment, transportation materials and equipment, and HVAC materials and equipment, water filters to remove microbial contaminants, water storage tank coatings as for use for ships, submarines, and space exploration vehicles to prevent biofilm formation and PVC piping to prevent biofilm formation. Within certain embodiments of the invention, methods for producing fine particulate organic ion exchange polymer salt materials are described, allowing for biological activation of the polymer salt as accomplished by ionic association of the organic ion exchange resin with a biologically active ionic agent. According to these methods generally, particles of a water-insoluble polysulfonated, polycarboxylated, polyaminated, or polyphosphorylated organic polymer salt or acid form of the material, for example a polymer salt of a cross-linked, functionalized organic resin, are combined with a biologically active ionic agent in an aqueous medium under conditions to facilitate substitution of the biologically active ionic agent onto the organic ion exchange resin polymer salt material by salt-exchange displacement of its counter-ion (e.g., a sodium ion) initially associated with the organic resin. This yields a biologically active ion exchange polymer salt particle having the biologically active ionic agent ionically associated with the organic ion exchange polymer salt material. According to the teachings herein, the biologically active ionic agent is thus rendered insoluble and will not freely dissociate from the biologically active polymer salt material in deionized water.

In another embodiment, the sulfonated polymer is not in salt form but the sulfonic acid form. This is added to a material to provide the local environment with low pH (high pH drop). This additive is best suited for use in polymers that cannot hydrolyze in the event of exposure to water. The materials most suited include polyolefins to include polyolefin elastomers, polyolefin blends, vinyl polymers and the like. Examples include polypropylene, polyethylene, polyvinylchloride, polystyrene, copolymers (to include block polymers) of styrene and butadiene and their hydrogenated counterparts, and styrene and isoprene for example.

A wide array of biologically useful ionic or ionizable drugs, compounds and other active agents can be employed to form the biologically active ion exchange polymer salts of the invention. In exemplary embodiments, the biologically active ionic agent is an antimicrobial agent. Suitable antimicrobial agents include ionic or ionizable antibiotics, antiseptics, antivirals, antiparasitic, and antifungals, oligodynamic metals, and ions capable of reactions to form reactive oxygen species (ROS). In other exemplary embodiments, an oligodynamic metal selected from silver, copper, zinc, iron, gallium, or bismuth is employed. In other exemplary embodiments, a cationic antibiotic is employed. Exemplary cationic antibiotics include tetracyclines or anthracyclines and aminoglycosides. In more detailed embodiments, a tetracycline is selected from tetracycline, doxycycline, minocycline, oxycycline, lymecycline, or apicycline, or combinations thereof and aminoglycosides include gentamicin and/or tobramycin. In yet additional exemplary embodiments, a cationic antiseptic is employed. Exemplary cationic antiseptics may comprise a guanidinium group (e.g., as exemplified by chlorhexidine or polyhexamethylenebiguanide), or a quaternary ammonium group (e.g., as exemplified by chlorhexidine, benzalkonium, cetylpyridinium, and cetrimonium (cetrimide)). Phosphorylcholine, a quaternary ammonium compound, by virtue of its molecular design, improves surface biocompatibility and lowers the risk of causing inflammation or thrombosis. This molecule is responsible for this important aspect of biomimicry. It is used commonly for medical device surfaces where anticoagulation is needed. Because of its structure that comprises an ammonium ion moiety, Sodium choline phosphate is readily attached to strong cation and weak cation exchange resins. With attachment and milling, phosphocholine modified resins can be generated by exchange of Phosphocholine chloride sodium salt using either the sodium form of strong and weak cation exchange resin sodium salts or the acid form of a strong exchange resin (—SO₃H). Coating materials modified with this compound can be used for devices in contact with blood to include catheters, stents, and heart valves.

In more detailed aspects of the invention, novel biologically active polymer “composites” and methods for preparing these composites, are provided. In exemplary embodiments, the biologically active composites are made by first providing an ion-exchange polymer salt, as summarized above. The organic ion-exchange polymer salt is typically a water-insoluble polysulfonated, polycarboxylated, polyaminated, or polyphosphorylated polymer salt. In exemplary embodiments the particles have a porous construction (macroporous), with individual particles defining channel, void and pore space surrounded by walls and partitions of polymer salt material. The ion exchange polymer salt particles are combined with a biologically active ionic agent in an aqueous medium to substitute the biologically active ionic agent by salt-exchange for a counter-ion initially associated with the ion exchange polymer salt material. This yields a biologically active porous ion exchange polymer salt particle having the biologically active ionic agent ionically associated with the ion exchange polymer salt material. By virtue of this novel preparation method and construction, the biologically active ionic agent is rendered insoluble, in that it will not freely dissociate from the insoluble ion exchange polymer salt material in deionized water.

In another exemplary embodiment, the particles have reduced porosity (microporosity) by virtue of their reduced cross link density. Such resins are known as “gel” or “gellular” resins that tend to absorb more water than their macroporous counterparts.

Following synthesis of the active ion exchange polymer salt material, the material is dried to remove most, or all, of the water present (e.g., water or an aqueous solution such as an alcohol). The biologically active ion exchange polymer salt particles are then milled by a high energy milling process.

The resultant fine particulate biologically active ion exchange polymer salt material is optionally blended with thermoset or thermoplastic or photocuring polymer precursors to form a fluid or semi-solid thermoset or thermoplastic or photocuring polymer composite mixture. This mixture comprises the fine particles of biologically active ion exchange polymer salt thoroughly or incompletely admixed with the polymer precursors (e.g., to form homogeneous or heterogeneous dispersions, or to blend the polymer salt particles only through a discrete portion of the composite mixture). After blending to a desired degree of mixing, thermoset or thermoplastic or photocuring polymer precursors are hardened or cured to form the biologically active solid polymer composite comprising the finely particulate biologically active modified polymer salt integrated within the thermoset or thermoplastic or photocuring or room temperature vulcanizing polymer. This new material is a solid biologically active polymer composite.

Any biologically acceptable thermoset, thermoplastic, room temperature vulcanizing or photopolymer can be employed within these aspects of the invention or in the case of aspects not requiring biologically acceptable polymers, industrial grade materials are acceptable. In exemplary embodiments, the thermoset or thermoplastic or photocuring polymer is selected from the group consisting of polysiloxane, polyalkylene (polyolefin), polyamide, epoxy, polycarbonate, polyester, vinyl, acrylic, polyurethane, polymers and combinations thereof. In certain embodiments, the polymer precursors comprise non-vulcanized (uncured) silicone rubber precursors.

When free radical vulcanizing silicone rubber precursors are used, these can be combined to form highly-adhesive silicone gels or fluids/liquids that are particularly useful in certain manufacturing methods and products of the invention. Silicone polymer composites can be cured under a range of conditions, for example at about 150 degrees for 5 to 10 minutes or with the addition of an appropriate photoactive catalyst, the polymer may be cured by exposure to UV radiation (e.g., using Momentive Performance Materials). Room temperature free radical vulcanizing and acetoxy-curing silicones can also be employed. Within more detailed embodiments, the biologically active ionic agent is an oligodynamic metal and the activated fine particulate product incorporating the oligodynamic metal is blended with silicone gel, liquid, or high consistency rubber further comprising an oligodynamic results in an earth-tone colored silicone product.

Also provided within the invention are materials and composites made according to the foregoing processes, and articles and devices incorporating these materials and composites. In certain aspects, biomaterials, products, tools and equipment are made that incorporate a fine particulate, biologically active ion exchange polymer salt or resin material as described. In more detailed aspects of the invention, biologically active, stable polymer composite materials are provided that comprise a fine particulate polymer salt ionically associated with a biologically active ionic agent, where the polymer salt is dispersed within a thermoset or thermoplastic or photocuring polymer to form solid, biologically active polymer composite. Biologically active polymer composites of the invention remain intact and biologically active without substantial chemical degradation, oxidation, hydrolysis, chemical decomposition, or photodegradation of the integrated ionic biologically active agent (e.g., wherein the biologically active agent remains stable and retains most if not all of its biological activity during preparation of the ion exchange polymer salt, and preparation and hardening/curing of the thermoset or thermoplastic or photocuring polymer).

Although additional novel aspects of the invention include the provision of novel materials and methods for producing “self-regenerating” or “renewable” materials and polymer composites by burnishing the surfaces, detailed studies have shown that the composites possess resilient and durable performance characteristics as demonstrated from studies involving repeated washings of painted (architectural) surfaces with CDC recommended disinfectants or for long duration exposure of a urinary catheter material to urine, known to possess high sodium content that will “drive” ion exchange. The forgoing and additional objects, features, aspects and advantages of the present invention will become apparent from the following detailed description.

Useful antibiotics include, but are not limited to, antibiotic molecules that are readily protonated to cationic form (each of which can be readily associated with either polycarboxylated, polyphosphorylated, and/or polysulfonated ion exchange polymers). Exemplary antibiotics include gentamicin; clindamycin; and doxycycline, among others.

Exemplary antiviral agents include, but are not limited to, acyclovir, idoxuridine, etravirine, and tromantadine, quaternary ammonium ions, copper ions.

Exemplary antifungal agents include, but are not limited to, copper ion, copper ion in combination with quaternary ammonium ions, zinc ion, zinc ion in combination with quaternary ammonium ions, miconazole, ketoconazole, fluconazole, itaconazole, econazole, terconazole, oxyconazole, grisefulvin, clotrimezole, naftifine, and polyenes such as amphotericin B or nystatin/mycostatin.

Exemplary antihistamines include, but are not limited to, diphenyl-hydramine, chlorpromazine, pyrilamine and phenyltoloxamine.

Useful antioxidant counter-ions include, but are not limited to, glutathione, cysteamine, and carnosine.

One important and unexpected realization of this invention requires an understanding that just because a fine particulate biologically active ion exchange polymer salt is (alone) active in a way expected by virtue of the known activity of ion bound to the resin (polymer) backbone, there are always high levels of uncertainty about whether a composition polymer (composite) resulting from the inclusion of a fine particulate biologically active ion exchange polymer salt will display activity. One case in point involves the inclusion of Cu(II)-SCER into epoxy and a composition resulting from its inclusion into silicone rubber. While the epoxy demonstrated strong efficacy against a variety of bacterial organisms, the silicone composite was completely ineffective for the same concentrations of Cu(II)-SCER across the same range of organisms, while the Ag-SCER silicone derivative was highly effective. Another important realization of this invention is that the speed (rate) at which a particular surface will kill a microorganism or inactivate a viral pathogen is an unknown and cannot be predicted. Although standard assay techniques (ASTM E2180 and ISO 22196) generally measure antibacterial effectiveness over 24-hours, others in the field have made efforts to demonstrate levels of efficacy over shorter periods (e.g., 2-hours) using real world conditions such as for a painted surface (25° C., 60% RH per U.S. EPA, per Sherwin Williams_Efficacy Review for Sanitizer #1; EPA File Symboi67603-RE; DP Barcode: D424257). Thus, if a novel composition performs better in a shorter time period as compared to an example from the published literature, such an outcome would not be anticipated or obvious. Therefore, this uncertainty provides a basis for why the measured outcomes presented herein would not be obvious nor could be anticipated prior to synthesis, processing, formulation, and testing.

### DETAILED DESCRIPTION OF EXEMPLARY EMBODIMENTS OF THE INVENTION

Described herein are compositions of polymeric ion exchange materials modified to include/incorporating biologically active ionic agents to form novel, biologically active ion-exchange polymer salts. These materials are useful for a variety of purposes, including as stable biologically active constituents of uniquely functional solid polymer composites. The activated or derivatized ion exchange polymer salts can be combined with thermoplastic or thermoset polymer precursors to generate biologically active polymer composite mixtures, including moldable, extrudable, layerable and paintable, activated polymer composite mixtures. These mixtures can be hardened or cured to make uniquely surface activated hardened polymer composite materials, coatings and components of textiles, devices, furnishings and apparatus, among other products.

Primary compositions of the invention comprise activated ion exchange materials typically provided in the form of polymer salts, including activated salts of organic polymer resins (insoluble cross-linked polymers). Suitable ion exchange polymers include cation exchange polymers as well as anion exchange polymers. The polymer salts incorporate one or more biologically active ionic agents, for example an ionic or ionizable antimicrobial agent (for example an ionic antimicrobial such as an oligodynamic metal, or ionic antibiotic, or an antimicrobial converted to an ionic form by chemical modification.

A wide assemblage of ionic or ionizable antimicrobial agents can be incorporated in activated polymer salts of the invention, including antibacterial drugs, antibiotics, antiviral agents, antifungal agents, organometallic compounds, and oligodynamic metals. Other useful biologically active agents within the methods and compositions of the invention include antiseptics, antimycotics, anti-inflammatory agents, antiproliferative agents, antineoplastic agents, chemotherapeutic agents, antihypertensive agents, anti-arrhythmic agents, anticoagulants, antioxidants, antiparasitic agents, anticonvulsant agents, antimalarial agents, amine-containing pharmaceutical agents, and other therapeutic agents obtainable in ionic form for use within the compositions and methods of the invention.

Biologically active ionic or ionizable agents are captured or bound in an insoluble matrix by ionic association with ion exchange polymers, often cation or anion exchange polymer “resins.” Useful derivatized ion exchange polymers may demonstrate insolubility in non-ionic aqueous media (e.g., distilled water). For example, polystyrene sulfonate when paired with a quaternary ammonium ion such as the benzalkonium ion, renders the resulting polymer poorly soluble in water yet soluble in organic solvents such as alcohol. This allows for the composition to be coated onto substrates to include fabrics or solid substrates such as fixtures, touch surfaces, counter tops and furniture. In some embodiments the ion exchange polymer may be insoluble or poorly soluble in non-ionic and ionic aqueous media. Associated with aqueous solubility, the subject ion exchange polymers often possess hydrophobic character. Polystyrene sulfonate is a polyanion with known antiviral activity. Polyanions can exhibit potent antiviral activity in vitro and it may be attributed to the inhibition of virus-cell fusion. As such, pairing the polystyrene sulfonate with a known organic antiseptic ion or oligodynamic metal could improve the activity.

Useful cation exchange materials for constructing biologically active polymer salts may include weak or strong cation exchange materials. Weak cation-exchangers may contain, for example, carboxyl (—CO2-H+) functionalities (alternatively “moieties,” or terminal or side functional groups). Strong cation exchangers incorporate sulfonate groups (—SO3-H+). In general, carboxylates (weak exchangers) are ionized over only a limited pH range, while a “strong” exchangers show slight variation in ion exchange capacity with changes in pH. Accordingly, carboxylates may exchange, release or allow dissociation of dications such as copper (II) much slower than sulfonates in a neutral pH medium where the presence of cations only will drive exchange.

In illustrative embodiments of the invention, strong cation exchange materials are constructed comprising polysulfonated salts of polymerized styrene (polystyrene). In other illustrative embodiments, polyphosphorylated materials such as cellulose phosphate or phosphates of synthetic organic structures are constructed. These polymeric ion exchange materials, such as those based upon polystyrene, may be cross linked with divinylbenzene to form insoluble styrene-divinylbenzene copolymer materials with varying degrees of solubility and hydrophilicity (water loving character) depending upon the amount of cross-linking agent included. These materials can be crosslinked to form insoluble ion exchange materials. Exemplary cross-linking agents include, but are not limited to, diacrylates to form acrylic-co-diacrylate copolymers or divinyl compounds such as divinylbenzene to form acrylic-co-divinylbenzene copolymers. Weak cation exchange materials are also provided, exemplified by polycarboxylic acid materials (salts or protonated forms) that may be acrylic structures formed by polymerization of acrylate materials. In alternative embodiments, cation exchange materials can include a diverse selection of polymers and functionalities, including styrene, acrylic, vinyl, sulfonate, carboxylate, and phosphate, among others. A variety of initial counter cations can be associated with the ion-exchange polymer base or scaffold, including for example sodium ions, potassium ions, and hydrogen ions.

Thus, in different exemplary embodiments of the invention cation exchange resins are primarily functionalized as polysulfonated salts, polycarboxylated salts, or polyphosphorylated salts. In some embodiments, the ion-exchange polymer will include two or more different polymer salts. Exemplary ion-exchange polymer mixtures include blends of polysulfonates, polycarboxylates, or polyphosphonates. These can be biologically active by salt exchange according to the methods herein with any of a diverse selection of cationic biologically active agents, for example oligodynamic metal cations, organic cations, or mixtures of organic cations and metal cations.

Anion exchange materials can include strongly basic or weakly basic anion exchange materials. Strongly basic anion exchange materials generally include poly (quaternary ammonium ion) salts and weakly basic anion exchange materials generally include polyamines that are generally secondary amine structures but can include tertiary amines as well. These ion exchange materials can be copolymers of styrene and divinylbenzene, sometimes referred to as styrene-divinylbenzene copolymers. In some embodiments, anion exchange materials can include polymers such as styrene, vinyl, amine, quaternary ammonium as well as counter anions such as chloride ion, hydroxide ion or carboxylate ion, for example.

The anion exchange or cation exchange materials may be functionalized as described and ionically bound to one or more biologically active ionic agents that possess a distinct biological activity (which may comprise a specific therapeutic efficacy, such as an antimicrobial or anti-inflammatory activity). Useful biologically active ionic agents include any biologically active agent (e.g., an antimicrobial or anti-inflammatory agent) that can be prepared in an ionic form, such as an ionizable salt form. The biologically active agent is loaded onto the ion exchange polymer typically as a substitute counter-ion by ion exchange to replace an initial counter ion (e.g., Na+) and form a new, biologically active polymer salt. The biologically active replacement counter ion can include any of a diverse selection of ionic or ionizable agents having a desired biological or therapeutic activity, including for example one or more of a metal cation, quaternary ammonium compound, organic ion, protonated amine, carboxylate, phosphate, cationic or anionic surfactant, and/or a biguanide. In exemplary embodiments, the counter-ion material can include one or more mono, di, and/or trivalent cation(s). Exemplary metal cations include, but are not limited to, Na+, Ag+, Au+, Cu++, Ga+++, Zn++, and Ce+++, Fe++, and/or combinations thereof. Exemplary quaternary ammoniums include, but are not limited to, benzalkonium (chloride), cetrimonium (cetrimide) (chloride), didecyldimethylammonium (chloride), and cetylpyridinium (chloride), noting that when incorporated into/onto the resin backbone, the cation is paired with one of sulfonate, carboxylate, or phosphate for example.

Exemplary protonated amines include, but are not limited to doxycycline hydrochloride, minocycline hydrochloride. Exemplary biguanides include, but are not limited to chlorhexidine diacetate, metformin, proguanil, and chlorproguanil.

Exemplary combinations/pairings of metal cations and quaternary ammonium ions include but are not limited to Cu++ and benzalkonium or Cu++ and cetylpyridinium. It has been shown that synergies between copper ion and quaternary ammonium ions can exist thus enhancing activity.

Useful biologically active cationic and anionic agents for binding to ion exchange polymer materials include, but are not limited to, antimicrobial compounds including oligodynamic metal ions, charged pharmaceutical agents including therapeutic agents or drugs effective in the treatment and care of multicellular organisms, and other ionic substances that can be improve the improve a particular clinical or biological environment. Among exemplary antimicrobial agents illustrated here are antibacterial drugs, including antibiotics, antiviral agents, anticoagulants, antifungal agents, organometallic compounds, antiparasitic drugs, as well as oligodynamic metals. Exemplary therapeutic agents include, but are not limited to, anti-inflammatory agents, chemotherapeutic agents, antibiotics, antioxidants, antimalarials, contraceptive agents including spermicidal agents, amine-containing pharmaceutical agents and the like.

Useful ion exchange polymer materials for association with biologically active ionic agents may be soluble or insoluble. The soluble versions of ion exchange materials include polystyrene sulfonate, vinyl sulfonate, as well as other versions of sulfonated linear polymers such as poly(2-acrylamido-2-methylpropane)sulfonate. In some embodiments, the ion exchange polymer material is an insoluble matrix or support polymer, which can take the form of small particles or beads of a few millimeters in diameter. Exemplary organic ion exchange resin materials of this type desirably possess porous particulate structures, with pores on the surfaces and channels and voids communicating with the surfaces of the resin particles. This porous construction enhances ion exchange functionality of the resin particles (i.e., it increases ability of the particles to communicate with and exchange biologically active ions for original counter-ions associated with the resin material from which the particle is formed).

Exemplary organic ion exchange polymers for use within the invention include monomers of one or more of styrene, acrylic acid, vinyl acetate, methacrylic acid, divinyl benzene, and/or butadiene, among others. In certain embodiments, the ion exchange polymer is cross-linked to modify solubility and ion exchange potential. Exemplary non-crosslinked polymers include, but are not limited to, poly(arylenevinyl)sulfonate, polystyrene-sulfonate, polyvinylsulfonate, polyalkylenesulfonate, polyantholesulfonate, and/or acrylamidomethyl propane sulfonate polymer. In one exemplary embodiment, a polystyrene is employed that is variably or adjustably crosslinked through addition of 0.1-55 mole % of divinylbenzene during polymerization—to create a range of selectable strength ion exchange capacity, loading potential (i.e., selectable total load capacity of biologically active counter-ion) and optionally a variable potential for dissociating the biologically active ion for drug delivery purposes (e.g., when in contact with physiological, ionic fluids and tissues). In some embodiments, the oligomer or polymer can include repeating units of the same monomer or a plurality of different monomers. The oligomer may be copolymerized with monomers and/or other oligomers to form a co-polymer or the linear polymers may be used without crosslinking. For example, the polymer backbone of polysulfonated cetylpyridinium salt may be polyarylenevinylsulfonate, polystyrene-sulfonate, polyvinylsulfonate, polyantholesulfonate, and/or acrylamidomethyl propane sulfonate polymer. In other embodiments a co-monomer such as divinylbenzene may serve to crosslink the polymer to increase stability and decrease solubility or hydrophilic character.

In exemplary constructions of activated ion exchange polymer salts, the initial ion exchange polymer may be selected from a commercially available polymer, for example a commercially supplied polysulfonated resin, such as Amberlite® IRP69 (Sodium Polystyrene Sulfonate-co-divinylbenzene USP, an insoluble, strongly acidic, sodium form cation exchange resin supplied as a dry fine powder) or Amberlite® IR88F (Polacrillin Potassium NF, a weakly acidic potassium form cation exchange resin supplied as a dry fine powder).

Ion exchange polymer materials for use within the invention are generally functionalized to bind or tightly associate ionically with cations or anions. For example, acrylics, styrenes and polyalkylenes may be functionalized by binding to one or more sulfonate, carboxylate and/or phosphorylate ions—to form such exemplary useful polymers as arylenevinyl sulfonate, styrene sulfonate, vinyl sulfonate, or divinyl benzene. These polymers will typically be employed in a first (unactivated) polymer salt form, lacking the “biologically active ionic agent”, and instead having an inactive, “initial counter-ion” present to exchange with the biologically active agent, such as sodium (Na+).

Functionalized ion exchange materials are often provided in the form of a “ion exchange polymer salt”, for example sodium polystyrene sulfonate. Illustrating general “salt exchange” designs contemplated here, the Na+ ionic component of the first polymer salt (sodium polystyrene sulfonate (a polymetallosulfonate)), can be exchanged with any of a variety of biologically active (e.g., antimicrobial or therapeutically effective) metal cations to prepare mono, di, tri, and even tetravalent metal ion, “biologically active polymer salt” derivatives. Similarly, polymetallosulfonates such as sodium polystyrene sulfonate, or polystyrene sulfonic acid can be converted to a polyorganosulfonate derivative (e.g., by exchange of sodium for any nitrogen atom containing salt/protonatable nitrogen compound of interest). Exemplary nitrogen atom containing salts/protonatable nitrogen compounds for use in these aspects of the invention include amines, ammonium ions, quaternary ammonium ions, amidines, amidinium ions, imines (iminium ions), thiazoles, imidazoles, guanidines, guanidinium ions, and/or pyridines, and pyridinium ions. In other illustrative embodiments, ammonium ion exchange polymer salt derivatives can be produced by exposing amino compounds to acid forms of polymers, for example and acid form of polysulfonate. In yet additional illustrative embodiments, metal dication derivatives can be produced by exposing acid or salt forms to divalent metal salts. For example, the addition of Cu(II) to the sodium form of poly(co-methacrylic acid-divinyl benzene) to yield the Cu(II) salt of poly(co-methacrylic acid-divinyl benzene) has been shown to be effective for the purification of bacteria from tap water. Normally present at concentrations as high as 500 CFUs/mL, the bacterial counts of tap water can be reduced well below these concentrations by simply passing the stream across a bed of the resin. Applications for the purification of water include dental water for irrigation use during dental procedures. Commonly, dental unit water lines are susceptible to the formation of biofilms on the lumens of the supply tubing because of the extended dwell times that tubing can hold stagnant water. Because of this, the lines are routinely flushed with special antimicrobial flushing solutions to include bleach, hydrogen peroxide, and silver citrate (Citrisil) for example.

In exemplary constructions of activated ion exchange polymer salts, the initial ion exchange polymer may be selected from a commercially available polymer, for example a commercially supplied polysulfonated resin, such as Amberlite™ IRP69 (Sodium Polystyrene Sulfonate-co-divinylbenzene USP, an insoluble, strongly acidic, sodium form cation exchange resin supplied as a dry fine powder) or Amberlite™ FPC88 H a strong cation exchange resin in acid form or Amberlite™ IRP88 (Polacrillin Potassium NF, a weakly acidic potassium form cation exchange resin supplied as a dry fine powder).

Insoluble ion exchange materials can be created by cross-linking. This may also be accomplished by combining a water-soluble ion exchange polymer with a dication such as a diammonium ion or a metal dication. At lower levels of cross linking (produced with a lower concentration of cross-linking agent), the polymer may possess some hydrogel or gel-like character, whereas at higher crosslink densities the absorption of water is minimized.

In certain aspects of the invention, ion exchange polymer salts are provided in particulate form before activation by salt exchange (i.e., to exchange the initial, inactive counter-ion with a biologically active ionic agent to form the activated ion exchange polymer salt). Suitable particles sizes of ion exchange polymers for preparation of activated polymer salts by salt exchange (to form biologically active ion exchange polymer salt particles) will often have an average particle size or diameter in a range of a conventional organic ion exchange resin, for example from about 0.0005 mm to about 2.5 mm, about 0.0005 mm to about 1.5 mm, or about 0.075 mm to about 0.5 mm. In other embodiments, the particle size diameter of the ion exchange polymer starting material will be from about 300-500 μm, or about 500-700 μm.

Exemplary ion exchange polymer salts employ a polymer matrix that renders the polymer effectively insoluble in water when crosslinked, whereas if the ion exchange polymer were not crosslinked a high degree of solubility could be obtained. Insolubility as used herein means that essentially all of the subject polymer material remains insoluble (e.g., precipitated) in deionized water at room temperature. Generally, the polymer matrix will remain insoluble even in ionic solutions, such as saline or physiological fluids. In illustrative embodiments, sodium polystyrene sulfonate and poly(vinyl carboxylic acid), otherwise known as polyacrylic acid, sodium salts are water soluble materials. These and like materials can be rendered more, or less insoluble for use within different aspects of the invention by adjustably or selectably _{[DV1]}varied degrees of cross-linking or ion pairing, as described herein.

As shown in illustrative Reaction Scheme 1, the number of positive charges in the depicted strong cation exchange polymer material is equivalent to the number of sulfonate groups present in the exemplary polymer. Accordingly, if the cation is a di-cation for example, it can be associated with more than an individual sulfonate, carboxylate or phosphorylate group.

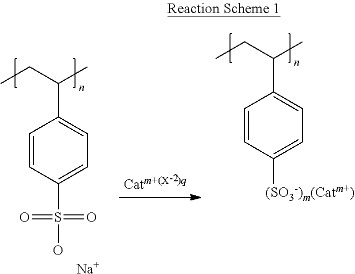

In other exemplary embodiments, polystyrene sulfonic acid-co-divinylbenzene is combined with the acetate salt form of an organic or metal cation (e.g., silver acetate, lithium acetate, zinc acetate or copper (I) or copper (II) acetate) in deionized water. The byproduct odor of acetic acid is evidence that the reaction has proceeded to yield the metal or organic sulfonate. The same reaction is also readily carried out using water soluble polystyrene sulfonic acid, the non-crosslinked version of the polymer. Cu(II), Zn(II), Ag(I), Ce(III), Ga(III) and a host of other derivatives are readily prepared from the corresponding acetate salts. The copper salt of polystyrene sulfonic acid is water soluble and can be used as an agricultural fungicide that can adhere to plant surfaces by spraying of dilute solutions onto trees for example. With drying of the aqueous solution, a thin film is formed. Similarly, a dilute solution in water or alcohol can be deposited as a coating onto non-woven (fabric) air filters or personal protective equipment (PPE) fabrics with the coatings able to inactivate viruses. The propensity of sulfonic acid to protonate carboxylate is the driving force for exchange. Hence any carboxylic salt can be used provided that the cation is not too large or sterically hindered to be bound to the resin. In yet another embodiment, the acid form of polymethaerylic acid-co-divinylbenzene can be reacted with the acetate salt of a metal or organic cation to yield byproduct acetic acid and the metal or organic salt of the methacrylic acid copolymer. The biologically active, exchanged counter-ions can be “variably loaded” onto the polymer backbone to finely adjust surface activity properties of the materials and products herein, for example by associating a selectable load concentration or density of from 1% to 100% of the biologically active, substituting counter-ion with functionalizing ion (e.g., sulfonate) on the polymer backbone, leaving “non-loaded” functionalizing ions remaining associated with the original counter-ion (e.g., Na+). Furthermore, a polymer backbone may be modified to include more than one active ion, for example cetylpyridinium(+) and silver(+), or any other combination of multiple active ions identified herein.

The maximal loading of biologically active counter-ion onto selected ion exchange materials can range to 100% but may be 90% or less of a theoretical maximum ion exchange capacity of the subject ion exchange material in some instances. Within this range the materials and products of the invention can be finely tuned for selected levels of biological activity (depending mostly on the agent and specific biological activity being employed) by metered loading of the ionic active agent onto the polymer backbone. For different activities where a lower “dosage” or loading is desired (e.g., when the biologically active ionic agent is particularly potent, or perhaps toxic, or when materials may be used in contact with sensitive tissues), the loading may be reduced to a moderate or low level (e.g., 50-60%, or 25-45%, respectively, of a determined maximum loading capacity of the polymer). In addition, mixed ions may be used. For example, polystyrene sulfonic acid may be loaded to a 90-100% exchange capacity but may be adjusted to be loaded with an active percentage of silver, and a second percentage of cerium, for example (e.g., as a balancing or blocking loaded material to limit, meter or normalize silver loading).

Variable loading of ion exchange polymers with one or more biologically active counter-ions to make biologically active polymer salts often involves use of ionic salt solutions having selectable concentrations (higher for higher targeted loading), and use of other variable conditions (e.g., varying temperature and/or pH, use of other solvents in addition to water, addition of other salts, etc.) conventionally used for ion exchange. Also considered in this context are differences in ion exchange capacity, for example cation exchange capacity (CEC), of a selected polymer. CEC represents the maximum quantity of total cations of any class that a polymer is capable of (ionically) binding at a given pH value. CEC can be expressed as milliequivalent (mEq) of cation per gram or per 100 grams (mEq/g or mEq/100 g) of ion exchange material.

In some embodiments, the functionalizing ion in the ion exchange material may be carboxylate. As shown in Reaction Scheme 2

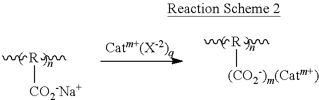

In some embodiments, the functionalizing ion in the ion exchange material may be phosphate. An exemplary cation exchange polymer of this type is cellulose phosphate. This material can be activated by antimicrobial cations such as copper (II), for example. Cellulose phosphate is a strong cation exchange material of variable ion exchange capacity (generally around 7 mEq/gram).

Biologically active counter-ions for activating ion exchange polymer materials can include any number of inorganic or organic cations or anions. Counter-ions that can be readily associated (without chemical conversion to an ionic or salt form) with useful ion-exchange polymers can include one or more metal cations, organic cations, quaternary ammonium compounds, protonated amines, carboxylates, phosphates, amine containing therapeutic agents, ammonium containing antibiotics and antimicrobial agents, nitrogen containing antibiotics and/or biguanides.

In some embodiments, the cationic or anionic biologically active agent is a mono, di, or trivalent metal including, but not limited to, an oligodynamic metal cation such as silver(I)/Ag(II), copper(II)/Cu(II), zinc(II)/Zn(II), iron(II)/Fe(II), gallium/Ga(Ill), cerium/Ce(ill), or bismuth(II)/Bi(II), amenable to ionic association with a sulfonate, carboxylate, or phosphate anion, for example. In other examples, the metal cation may be one or more of a monocation species Na+, Ag+, K+, Li+, Au+, a dicationic species Ba++, Ca++, Cu++, Zn++, Mn++, Mg++, Fe++, or a trication species such as Bi+++, Ga+++, and/or Ce+++ or combinations thereof. In illustrative embodiments, useful materials for association by counter-ion exchange with an ion-exchange polymer salt (e.g., a polysulfonated resin salt), include, for example, a silver salt, copper (II) salt, cerium (III) salt, Gallium (III) salt, cetylpyridinium salt, benzalkonium salt, chlorhexidine salt, centrimonium (centrimide) salt, octenidine salt, zinc (II) salt, iron (II) salt, or minocycline salt, or combinations thereof. Monocations require binding to a single sulfonate anion, dications require binding with two separate sulfonate anions and trications require an interaction with three sulfonate groups.

The sulfonate may also be associated with varieties of ammonium ions to include NH4+, RNH3+, R R⁻NH2+, RR R NH⁺, or ⁺, RRRR N+, for example where R represents an aryl, alkyl or mixed aryl alkyl group or the sulfonate can be associated with a pyridinium cation. According to another example, the sulfonate group can be associated with one or more of organic species including nitrogen containing organic species such as an amino acid, a tetracycline, doxycycline, arginine, gentamycin, ammonium chloride, cetyltrimethylammonium bromide, lysine, glutathione, lidocaine, albuterol, and/or alkyl/benzylammonium, pyridinium such as cetyl pyridinium, a guanidinium ion such as with chlorhexidine or polyhexanide, amino or oxazole, triazole, or thiazole containing compounds such as antifungal agents to include ketoconazole, or clotrimazole, and (dihydropyridinyl) species such as octenidine for example. In some embodiments, the material may be ionically bound to a plurality of therapeutically useful counter-ions. For example, an oligodynamic metal ion and a quaternary ammonium ion may both be bound to the same polymeric ion exchange material. In other embodiments, more than one therapeutically useful counter-ion from the same class may be bound to the same copolymeric ion exchange material, compositions described herein include, but are not limited to, antibacterial drugs, including antibiotics, antiviral agents, antifungal agents, organometallic compounds, antiparasitic drugs, as well as oligodynamic metals.

Useful antibiotics include, but are not limited to, natively cationic antibiotics and antibiotics that are readily protonated to cationic form (each of which can be readily associated with either polycarboxylated, polyphosphorylated, and/or polysulfonated ion exchange polymers). Exemplary antibiotics include gentamicin; clindamycin; and doxycycline, among others.

Exemplary antiviral agents include, but are not limited to, acyclovir, idoxuridine, etravirine, and tromantadine, quaternary ammonium ions, and copper ions.

Exemplary antifungal agents include, but are not limited to, copper ion, copper ion in combination with quaternary ammonium ions, zinc ion, zinc ion in combination with quaternary ammonium ions, miconazole, ketoconazole, fluconazole, itaconazole, econazole, terconazole, oxyconazole, grisefulvin, clotrimezole, naftifine, and polyenes such as amphotericin B or nystatin/mycostatin.

Exemplary antihistamines include, but are not limited to, diphenyl hydramine, chlorpromazine, pyrilamine and phenyltoloxamine.

Useful antioxidant counter-ions include, but are not limited to, glutathione and carnosine.

These and other aspects of the invention described herein may be clarified in reference to prior, related disclosures in PCT/US2015/049255, filed Sep. 9, 2015, based on U.S. Provisional 62/047,655, filed Sep. 9, 2014, and PCT/US2020/059909, filed Nov. 11, 2020, based on U.S. Provisional 62/934,510, filed Nov. 12, 2019, each incorporated herein by reference for all purposes.

In other novel embodiments of the invention, catheter hubs, the connector portion of the catheter that allows for the connection to a drug reservoir such as for chemotherapeutic delivery or a dialysis machine for purifying blood can be injection molded from biologically active polymer composites of the invention, for example using a matrix material selected from polycarbonate, acrylic or polypropylene and the septum is generally constructed of silicone rubber. The composite hub and septum may comprise an active copper (II) resin such as Cu(II)-SCER. In addition to the known antimicrobial effect of Cu(II) alone, the surfaces can be cleansed by the addition of dilute sodium chloride in water (saline or >0.9% NaCl, 154 mEq/L) and hydrogen peroxide as added directly to the hub using a disinfection cap inclusive of an attached sponge such as the 3M Curos® Disinfecting Port Protector comprising isopropyl alcohol. In this example, at neutral pH NaCl (aqueous) facilitates release of Cu(II) and hydrogen peroxide acts on the copper ion in a pseudo-Fenton reaction as described in equations (1-3): 1. Cu(II)+H2O2 Cu(III) HO—+OH⁻, 2. Cu(III)+H2O2 Cu(II)+HOO—+H⁺3.2H2O2+HO—+HOO—+H2O.

These embodiments achieve qualitative production of hydroxyl radical on the surfaces of epoxies, PVC, silicone and other materials per the inset diagram, e.g., using a methylene blue bleaching test (Satoh, A. et. al., Environ. Sci. Technol. 2007, 41, 2881-2887).

This approach to generating hydroxyl radical decontaminant may be used in other embodiments, for example to produce mouth pieces, dental materials and implants resistant to harmful biofilms, and for producing microbe-inactivating coatings, fabrics and the like for use in vehicles, ships, aircraft, submarines, tanks, plumbing and air circulation ductwork for buildings and ships, aircraft, submarines, tanks, clinical surfaces, tools and equipment, and to protect or decontaminate personnel exposed to biological contaminants, chemical warfare agents and other contaminants. Environments where this may be important include water storage and water-recycling/reuse systems found within the international space station vehicles for space travel, submarines, ships and the like where lightweight composites are used for air handling duct work that may be susceptible to infection and virus transmission for example, for example.

In certain embodiments, the use of a Cu(II)-SCER modified coating, such as by an epoxy

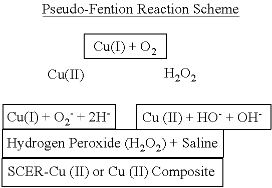

coating or epoxy composites, for example an Olin epoxy comprising a 2-part resin (DER 331)+Hardener (DEH 487 for example, or other polymer composite materials incorporating a Cu(II)-SCER or Cu(II)-WCER will be sufficient to, safely and effectively, prevent contamination, biofilm formation, virus transfer and other adverse conditions. Notably, Cu(II) must be liberated from the coating for the pseudo-Fenton reaction to be effective. To accomplish this, some cations (e.g., Na+) are necessary. Drinking water is generally much lower in salts, e.g., Na+ than saline and as such the Fenton reaction will be much slower in the drinking water medium. However, this also is a benefit when it comes to longevity/durability.

In yet another embodiment, the tubing and face masks of a continuous positive airway pressure (CPAP) device may be formulated with Cu(II)-SCER and/or Cu(II)-WCER to enable simple and facile disinfection of equipment by exposure to a solution of saline and dilute hydrogen peroxide. In a similar embodiment the Ag-SCER additive may be used instead.

In one exemplary embodiment, a port of a central venous catheter (CVC) comprising a polycarbonate (female) luer connector fitted with a silicone rubber, or other appropriate rubber material septum and both components formulated to include fine particulate organic ion exchange powder salt in Fe(II) or Cu(II) form and at the time of pairing the female luer connector of the CVC with the male luer counterpart for the delivery of medicament or nutrition, the female portion of a luer connector is swabbed with a sponge containing hydrogen peroxide solution. The sponge may be fitted onto a male luer connector in order to allow the cap to be turned to rub/swab the hydrogen peroxide moistened sponge across the surfaces of the female luer connector thus enhancing fluid contact and the uniform generation of superoxide as a means of adequately disinfecting the inner surfaces of the connector. This embodiment is described a means of preventing catheter-related blood stream infections (CRBSIs).

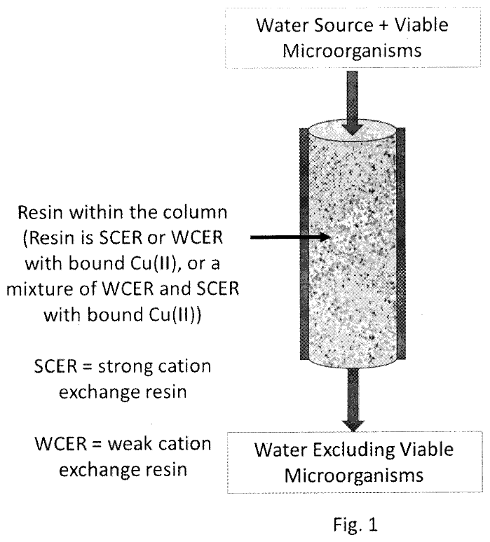

In illustrative water processing and storage systems, coatings that incorporate a fine particulate biologically active ion-exchange polymer salt material of the invention can be applied to stainless steel holding tanks, e.g., as epoxy coatings or other polymer coatings, which may include powder coatings, such as fusion bonded epoxies or polyesters. It has been demonstrated that methylene blue bleaching (indicative or hydroxyl radical production) can occur on the surface of epoxy modified to include 3 wt. % Cu(II)-SCER treated with a 1:1 mixture of 0.9 wt % NaCl solution and 3% H2O2, effectively. It is also possible to increase both the NaCl and hydrogen peroxide concentrations to optimize these compositions. These resins may be applied as “resin cartridges” that are provided in-line with a water source. Here water is pumped through a columnar cartridge charged with a Cu(II) salt of a weak and/or strong cation exchange resin and the system can be cleansed of suspended microbes that adhere to the resin surface, as depicted in FIG. 1.

In other exemplary embodiments, PVC sink drain piping (e.g., P-trap) or floor drains injection molded from PVC formulated to include a copper(II) ion exchange resin effectively prevent microorganism proliferation and biofilm formation, including for antimicrobial-resistant microorganisms found within clinical institutions (e.g., MRSA) and food processing facilities (e.g., Listeria monocytogenes sp. responsible for listeriosis). In order to disinfect, the drains can be treated with a mixture of saline and 3% (or stronger) hydrogen peroxide to initiate the Fenton reaction.

In a simple embodiment of a water purification device, water removed from a source (river, lake, spring, etc.) can be filtered through a high surface area (reticulated) filter fabricated from an active polymer salt particulate embedded within a polymer matrix (to provide an active polymer composite) into a container containing a high surface area sponge-like substrate fabricated from or coated with an active polymer salt particulate embedded within a polymer matrix. Bacteria that adhere to the surface will be killed immediately and those remaining in the water will be killed in time with exposure to the active surfaces.

In certain embodiments, the fine particulate polymer salts can be synthesized as biodegradable resins to permit release into the environment, or to permit safe intranasal or intrapulmonary delivery of active particulate aerosols to carrying therapeutic ionic active agents to target tissues of mammalian subjects, where the ionic agents may be released (dissociated and solubilized from the polymer salt carrier following contact with a physiological ionic fluid) or mediate surface active drug, antimicrobial or therapeutic activity.

In additional aspects of the invention, a wide range of orthopedic biomaterials and devices will beneficially incorporate activated polymer salts and polymer composites of the invention. Among many orthopedic uses contemplated are the posts of implants are known to be high-risk conduits for entry of microbial infectious agents into hip implant patients and orthopedic pins that are external to an affected site. The invention provides a variety of useful composites to prevent this contamination/infection risk, including epoxy, silicone and acrylic plugs, or acrylic bone cement compounded with sulfonated polystyrene-divinylbenzene-tobramycin or gentamicin salt (or polymethacrylic acid-divinylbenzene-tobramycin or gentamicin salt) for placement at a site of a hip implantation post. These composites and devices provide effective slow release of ionically associated drug over time. In more detailed embodiments, these composites (generally applicable for adjunctive use with a diverse array of prosthetic implants, including dental and surgical posts, pins, anchors, sutures, stents, etc.) are often formed as a porous solid composite (e.g., spongiform, lattice form, open cellular, blown or extruded composite), which can be facilitated by addition of any of a variety of known useful polymer foaming agents—to increase surface area for enhanced drug delivery (i.e., with faster kinetics or higher doses of drug delivered, and more effective sustained delivery, e.g., with effective delivery amounts maintained for 1-3 days or weeks, 1-3 months or longer). Polyurethane-based polymer composites described herein are particularly amenable to fabrication of foams. The pores are the result of the liberation of CO2 resulting from the addition of water during the cure process.

The attachment of surface modifying compounds to ion exchange polymers can be readily accomplished by first converting the resin to its acid chloride and subsequently reacting the resin with a long chain fatty amine such as octadecylamine. Generating powder from the modified resin and incorporating the powder into a polymer can alter the surface hydrophobicity significantly. These attachments can be created through sulfonates, phosphates or carboxylates. When anion exchange resins are employed, carboxylates, sulfonates, phosphates and other anions can be bound.

Additional embodiments of the invention provide effective hemostat compositions that prevent and/or stop bleeding. Exemplary hemostat compositions employ epinephrine, a hormone and neurotransmitter produced by the adrenal glands, exchanged onto the backbone of a strong or weak cation exchange resin rendered as a fine particulate as described and loaded into a textile or foam compression dressing for application to a bleeding wound. In related embodiments, anesthetics such lidocaine, novocaine, mepivacaine, tetracaine, benzocaine, bupivacaine, ropivacaine, or articaine are also loaded onto the resin backbone and incorporated into the polymer matrix, textile, foam or other material or device for therapeutic application onto or within wounds (e.g., traumatic or surgical wounds, burn sites, etc.). The anesthetic will improve efficacy and tolerability of hemostatic and antimicrobial dressings and enhance local pain management.

In additional hemostatic embodiments, calcium salts of a strong or weak cation exchange resin (in powder form) can be combined into a hydrophilic large pore, high surface area open cell foam and resulting dressings deployed as hemostatic devices. Blood coagulates in pores of the dressing material or device, resulting in clotting and stoppage of blood flow.

In an alternative embodiment to a hemostat, a strong anion exchange resin such as Purolite A502PS is milled to small particle size and added to a polyurethane foam matrix to allow for the preparation of a thin flexible sheet that can be pressed into a wound to prevent blood loss. The quaternary ammonium groups interact with negative charged sites on membranes of erythrocytes, mediating bio adhesion that facilitates coagulation and achieves hemostasis. In each of the foregoing hemostatic embodiments, a range of forms can be employed as described for the production of wound dressing, coatings, compresses or other hemostatic devices, for example including woven and non-woven materials (e.g., spunlaid and melt-blown non-wovens), polypropylenes and a variety of other materials in the form of solids, fabrics, films, foams, laid, sprayed or applied coatings, and the like. In exemplary embodiments, polyurethane foams incorporating antimicrobial and/or hemostatic ion exchange resins can also be utilized for beds and pillows in hospitals, nursing homes, corrections institutions, and other large-scale housing and care facilities.

Combining the particles of ion exchange polymer material with a salt comprising an antimicrobial cation, for example, in an aqueous medium will mediate salt exchange of the antimicrobial cation for the initial counter cation present on the ion exchange polymer—to yield an antimicrobially activated polymer salt derivative (having the antimicrobial cation ionically associated with the polymer). These salt-exchange processes can render the newly-associated, biologically active ion exchange salt effectively insoluble in water (i.e., the active counter ion will not freely dissociate in distilled water). The resulting antimicrobial polymer salts are broadly active against gram negative and gram-positive organisms to include several species (spp), fungi, and yeasts to include several species, and viruses to broadly include enveloped and non-enveloped viruses.

In exemplary embodiments, Cu(II)-SCER-modified acrylic latex enamel paint (2.0-3.0 wt % loading) demonstrated exceptional and surprising effectiveness against the respiratory (RNA) viruses of HCoV-229E tested at 2.0 wt %, and H1N1 influenza virus (a subtype of influenza A virus, also known as swine flu) tested at 3.0 wt %. In addition, the Cu(II)-SCER paint formulation at 3.0 wt %) was very effective against feline calicivirus, an RNA virus accepted as a predictive surrogate for testing anti-norovirus agents. Cu(II) is anticipated to inactivate both DNA viruses and RNA viruses as noted by Sagripanti et. al., whereby they tested the effectiveness of copper(II)chloride against 5-different species of single stranded and double stranded (RNA/DNA) viruses or phage. Although Cu(II) was effective, high concentrations (10-1000 mg/L, i.e., 10-1000 ppm or 0.157-15.7 millimolar concentrations) of Cu(II) were needed to be effective. With the addition of hydrogen peroxide (100 mg/L) the concentrations needed were greatly reduced (doi: 10.1128/aem.59.12.4374-4376.1993). In the compositions of the instant invention described, surface concentrations of the Cu(II) liberated are surprisingly sufficient to significantly reduce viral titers in a short period of time particularly considering that the concentration of Cu(II) in the final paint product ranges from 0.24-0.36 wt % Cu(II), 38 micromolar-57 micromolar concentrations, nearly 1/1000th the concentrations described as antivirally effective evaluated by Sagripanti et. al. The log reduction outcomes measured for these unique antimicrobial decorative and architectural coating compositions are unexpected in light of this significant concentration difference.

Addition of ionic copper-modified resins, e.g., (Cu(II)-SCER), to commercially available water-based architectural paints and other coatings has heretofore been problematic, based on the fact that copper induces formation of agglomerates within these commercial materials that resembles curdling. This is undesirable from a consumer perspective and correlates with problems in even and reliable application of copper-containing paints and coatings. This problem relates to a precipitation phenomenon, resulting from Cu(II) modified resins forming pseudo-crosslinks with ionic surfactants found in some conventional paints. One approach to this problem employed within the invention is to react the Cu(II)-SCER with an amine, for example ammonia vapor, to form a metal-ammine complex. In this complex, at least one mole of ammonia interacts with each atom of Cu(II). When the ammonia binds, the material undergoes a stark color change to a deep blue compound. With inclusion into paint comprising ionic surfactants, no curdling is observed. Subsequent coating of a surface and allowing the paint to dry facilitates evaporation of the ammonia from the unstable Cu-Ammine complex comprising ammonia (NH3). However, because ammonia is a toxic irritant, avoiding its use is desired in many consumer and institutional (e.g., hospital) applications. To address this related problem, Applicants have exhaustively surveyed available commercial coating products, whereby a useful subset of paint products was identified comprising acrylic latex enamel paints compatible with Cu(II)-SCER, likely comprising non-ionic surfactants), thus avoiding the physical (viscosity) changes initially encountered when addition of Cu(II)-SCER was attempted.

Within various embodiments of the invention, insolubility or non-dissociability of the active, ionic antimicrobial agent can be controlled to allow for partial solubility or dissociability (e.g., of active counter-ions from activated ion exchange polymer salts), for example by using weaker ion exchange materials, multivalent active counter-ion agents, and other methods. Thus, in certain embodiments of the invention, the biologically active counter-ion agent may be partially soluble in ionic aqueous media, or may be completely, reversibly associated with the ion exchange polymer such that it is insoluble in distilled water and other non-ionic media but rendered freely soluble in ionic media such as saline and physiological fluids. In this manner the biologically active polymer salts and related composites of the invention can function in multiple activity modalities. In primary activity modality, the activated polymer salts and composites exert their biological effects mostly as “surface activity”, where the biologically active ionic agent functions primarily at a surface of the polymer salt or composite, without appreciable dissociation (e.g., less than 2%, less than 5%, or less than 10% dissociation/solubilization after 1, 8, or 24 hrs.) of the active ionic agent from the surface.

In an alternative or combined modality, the activated polymer salts and composites can also exert “non-surface” biological effects as drug delivery materials or devices, wherein in addition to “surface activity” the biologically active ionic agent is also “reversibly-associated” with functional groups on the ion exchange polymer salt materials in the composites. They are therefore ionically dissociable from the composite surface under certain conditions and can be released in a soluble form following exposure to, e.g., ionic aqueous media including physiological fluids. In these aspects of the invention, polymer composites incorporating activated ion exchange polymer salts function as drug and active agent delivery materials and devices—i.e., to deliver dissociated, biologically active ionic agents to tissue and compartments adjacent to or distant from the polymer salt/polymer composite surface.

Generally, the surface area of the device is a significant factor in delivery (e.g., foams yield high surface areas, versus a lower surface area, textured or solid composite material). Surface area of different constructs can be controlled, for example by material choice, and by fabrication and molding techniques (such as spraying, coating, blowing, molding and extrusion techniques that include coextrusion). In certain embodiments it is important to restrict contact of an activated composite material with a surface (e.g., an inner lumen) or portion of a device the composite is being attached, layered or molded to. The hydrophilicity of the polymer matrix may also play a role in the surface release characteristics of materials and devices of the invention.

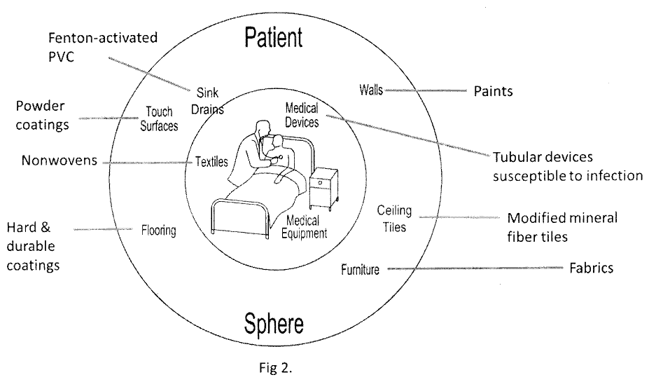

In more detailed embodiments of the invention, the fine particulate activated ion exchange polymer salt materials thus produced are useful in a wide variety of biomedical methods, compositions, materials, polymer composites, and devices including devices where a hydrophilic matrix (carrier) is employed. Such applications include hydrophilic coatings on the surfaces of medical devices such as catheters (tubing) and hydrophilic carriers such as in foams, sponges, biosensors, and sheet-stock materials that can be used in wound healing (vacuum-assisted closure), wound dressings including wound contact layers, vaginal sponges and hard plastic enclosures of medical equipment used within the hospital/clinical environment and the like as depicted in FIG. 2. Beyond medical devices, tubing plays a significant role in food handling and production and pharmaceutical preparations. As such, tubing materials of the invention are useful in these and other diverse industries, to prevent or minimize bacterial, fungal and viral contamination without excessive leaching of undesired or toxic byproducts. Of significant importance is the sulfonate carrier of the antimicrobial cation. Because carbonated beverages present pH values below 7 as a result of the presence of carbonic acid (HCO₃) or phosphoric acid (H₃PO₄) as in cola some materials will liberate antimicrobial cations such as Ag⁺ or Zn⁺⁺ at reduced pH. The resulting salts of these ions can alter taste at the ppb/ppt level and as such can present limitations to certain antimicrobial solutions. Because the conjugate acid of silver sulfonate (—SO₃Ag), (i.e., benzene sulfonic acid possesses a pKa of ˜−2.8), affixed to the resin cannot be protonated by carbonic acid (pKa₁=6.37, pKa₂=10.32) or phosphoric acid (pKa₁=2.16, pKa₂=7.21), pH is not a mechanism by which the silver ion can be released. Pure ion exchange is the only true means of release. This holds true in an experiment that placed a modified polyolefin elastomer in contact with seltzer and cola for 30-days at refrigerator temperature as no discernible difference in taste was notes nor were any precipitates observed in the test medium.

Tubing is incorporated within many medical devices including urinary catheters, ureteral stents, cerebral shunts, central venous catheters including peripherally inserted central catheters (PICC line), dialysis catheters, wound drainage catheters, endotracheal tubes, pacemaker and implantable cardioverter defibrillator lead bodies, and cerebrospinal shunts for example. Within these and related devices, an approach that is an alternative to making the entire lead body from an antimicrobial composite, the lead connector bundle (i.e., the over-molded part of the lead where the distal and proximal electrode connectors are immobilized, for both pacing and defibrillator leads) can be formulated to include a micronized antimicrobial strong or weak cation exchange resin. Endotracheal tubes as used for airway management can be wholly manufactured from plasticized PVC, polyurethane, or silicone Ag-SCER composites, including the retention cuff, a balloon that is filled with air and sits at the branch leading to the lungs to enable mechanical breathing through the main tube element. The retention cuff is the most problematic component because subglottic fluid build along the top of the retention cuff at the intersection of the retention cuff and the trachea, seepage of contaminated subglottic fluids into the lungs results in a high incidence of ventilator-associated pneumonia (VAP). Fabricating retention balloons and/or cuffs for any device from compositions containing antimicrobial strong or weak cation exchange resins can be an important improvement in devices of today to help eliminate the over-prescribing of antibiotics to prevent/treat infections. Antibiotic prescriptions administered for VAP and CAUTI in the ICU represent significant percentages of the overall use of antibiotic therapy in hospitals.

Biofilm-induced catheter-associated urinary tract infections (CAUTIs) result in considerable morbidity while contributing to large increases in the cost of health care. The composite materials described can be used to manufacture ureteral stents, urinary catheters, and urine drainage bags. All of these devices can facilitate/enable CAUTIs. Silicone, PVC and polyurethane composite biomaterials are effective when combined with Ag-SCER at loadings of 2.0 wt % or less.

A variety of bacterial species are known to colonize ureteral stents and subsequently form biofilms on the outer and inner surfaces of the devices. It is organisms that produce urease that facilitate the formation of (luminal and extraluminal) mineral-encrusted biofilms by its action on urea, degrading it to ammonia and carbon dioxide thus increasing the pH of the urinary tract. Higher pH leads to precipitation of Ca++ and Mg++ phosphates. The most problematic biofilms are crystalline in nature, and these can induce trauma to the bladder, urethra, ureter, and kidney epithelium with device removal. Also, crystalline biofilm debris can induce stone formation and blockage of urine flow when these biofilms are disrupted. If blockages go undetected, patients can suffer pyelonephritis and septicemia. In addition to utilizing antimicrobial ion exchange resin-modified composites to reduce or eliminate surface dwelling organisms, resins modified to include a urease inhibitor can be included. One such compound that can be bound to the resin is cysteamine (2-aminoethanethiol). Because ureteral stents are small devices, generally weighing only a few grams, antimicrobial coatings are unlikely to provide benefit given the presence of a small volume coating on a very small device which will limit the availability of active agent(s). Moreover, a coating on the inner lumen could jeopardize the patency of the device given the small size of the lumen. The invention overcomes these obstacles by providing ureteral stents fabricated from antimicrobial composites as described, for example silicone+Ag-SCER, wherein the entire device is constructed from an antimicrobial composite. In one alternative embodiment, the inner lumen maintains a significantly higher Ag-SCER concentration that the external (tissue contacting surface) in order to prevent microorganisms from proliferating and forming biofilm inside the device where urine passes from the kidney to the bladder.

Ureteral stents frequently cause flank pain in recipients. Antimicrobial ureteral devices of the invention may include an external (hydrophilic) coating comprising an anti-inflammatory agent, such as dexamethasone or ketoprofen, of low solubility, to provide long-term relief of pain and reduced infection. Corticosteroids in ureteral stented patients have been shown to be safe over many months at doses of about 30 mg daily. With the addition of these modified coatings, clinical use and effectiveness of the subject devices can be extended.

In certain embodiments of the invention, fine particulate activated ion exchange polymer salt materials are combined with other polymer materials to produce biologically active solidified polymer composites. The fine particulate ion exchange polymer salt is generally admixed in effective amounts with polymer precursors that can be fabricated into various constructs or the fine particulate ion exchange polymer salt is admixed with a coating solution for deposition onto a surface. One example application is for use as a (diagnostic) substrate when formulated to include IRP69-Na or other appropriate ion exchange resin chosen on the basis of the target analyte of interest. Such a substrate can be placed below ground, allowed to dwell for some period of time and subsequently harvested (removed from the ground) and the fabric analyzed for metal uptake (e.g., Cu(II), Fe(II), As(III, V for example) with the aid of atomic absorption (AA) or inductively coupled plasma (ICP) spectroscopy.

In other examples, inkjet technology can be employed to deposit an array of lacquers comprising one or more of a fine particulate activated ion exchange polymer salt materials onto the surface of a sacrificial probe that can be placed below ground, or in a wastewater stream, whereafter the probe is removed and the material analyzed for a variety of contaminates, including Pb, As, Sb, Cd and other toxic agents. Typically the ion exchange polymer for these applications is biodegradable.

Production of biologically active solid polymer composites of the invention is schematically depicted in manufacturing Reaction Scheme 3, where R is a group containing carbon and n is greater than 1.

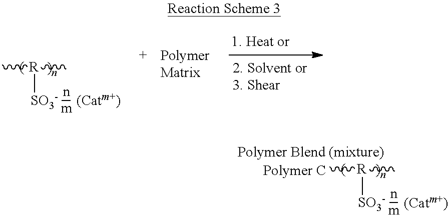

The polymer matrix (precursors) may be a polymeric composition that includes one or more useful polymer precursor types, for example from the group silicone rubber, polyurethane, a polyester, a polycarbonate, a vinyl polymer (PVC, PVA, PVAc, Polyvinylidene chloride, polyisoprene, SIBS, ABS, SBS, polystyrene, hydrogenated vinyl polymers, e.g., SEBS), a polyalkylene such as polyethylene, a polyamide, an epoxy, complex polymer mixtures to include paints, lacquers and coatings of polyurethanes, acrylics, PVC, epoxies, and any variety of other polymers, an acrylic, a cellulosic, a fluoropolymer, or a biopolymer such as collagen, hyaluronic acid, gelatin, a hydrogel polymer, and/or an alginate.

In some embodiments, the polymer precursors comprising the polymer matrix may be provided as one or more polymer precursors in a substantially unsolidified (fluid or semi-solid) state. Prior to solidifying the polymer composite, the precursors are blended with biologically active ion exchange polymer salt particles to form a polymer composite mixture. This mixture is then solidified to form activated solid polymeric composites, and related biomaterials and products.

In various embodiments, activated polymer composites are made with any of a diverse array of polymer precursors classified as thermoplastic, thermoset, elastomer, and/or rigid polymer precursors. Exemplary polymeric precursors include, but are not limited to, one or more of polyalkylene, polysiloxane, polyamide, epoxy, polycarbonate, polyester, polyol, polyarylene, vinyl polymer, acrylic polymer (polyacrylonitrile, polyacrylate, polymethylmethacrylate), asphalt, bitumen, polysaccharide, cellulosic, and/or polyurethane. Exemplary polymer precursors comprise nonvulcanized silicone rubber precursors. Primary products of the invention (biologically active ion exchange polymer salts) can be combined with a variety of thermoset or thermoplastic or photocuring polymer precursors to make solid composites having a range of biological surface activities (and optionally, non-surface drug delivery activity) and a commensurate array of applications and methods of use. Surface activation of the inventive polymer composites (i.e., specific biological activity or activity potential, exhibited by an exposed surface of the polymer composite) can vary depending on the type and identity of the biologically active ionic agent (bound to) incorporated into the ion exchange polymer salt, as well as on the amount and distribution of the active polymer salt within the hardened polymer composite.

In certain embodiments of the invention, the biologically active polymer salts and polymer composites are useful to prevent attachment, colonization and/or survival of microbes (e.g., bacteria, viruses and/or fungi) or other pathogens or parasites transmissible by surface contamination on a fomite or other targeted surface. To the extent colonization of a surface bearing an activated, antimicrobial polymer salt or polymer composite of the invention is subject to “contamination” by a subject microbe or other pathogen, the activated polymer salt or composite functions distinctly by reducing or preventing secondary transmission of viable pathogens to a vulnerable living subject, for example a veterinary or human patient in a clinical or home medical care environment as depicted in FIG. 2. By possessing activated, antimicrobial (e.g., bactericidal or bacteriostatic) surface activity, the invention either prevents or limits contamination, or reduces bacterial growth or viability on contaminated surfaces, such that when these surfaces are secondarily brought into contact with a living subject the rate of transmission or “infection” from an activated polymer surface to the subject (e.g., compared to a surface made of the same material and exposed to the same experimental contamination inoculum, not activated by incorporation of a biologically active polymer salt carrying the biologically active ionic (antimicrobial) agent).

These distinctly potent antimicrobial activities are readily demonstrated using conventional and standardized assays such as ASTM or ISO-based assays. For example, The ASTM E2180 assay or an ISO 22196 or modifications thereof. The assays can be used to evaluate 24-hour, 2-hour, 1-hour, or 0.5-hour antimicrobial effectiveness.

According to these methods, the active polymer salts and composites of the invention exhibit extraordinarily high levels of surface decontamination activity (e.g., bactericidal and/or bacteriostatic surface activity). This potent activity manifests within as little as 1-10 minutes after inoculation/contamination of these unique biomaterials. Within a half hour after surface contamination, or in some instances after from one hour to three hours, full expression of maximal surface decontamination activity is observed for many antimicrobially active polymer salts and polymer composites of the invention. In many instances this amounts to an effective total surface decontamination, where consistently no viable microorganisms remain viable or transferable from a contaminated surface after a post-inoculation activity expression period. These observed results are truly remarkable in comparison to contamination and transfer data observed from similarly treated control biomaterials (i.e., comparable ion exchange polymer salt materials not activated by association with biologically active counter-ions, or comparable polymer composites incorporating ion exchange polymer salt materials not activated with biologically active counter-ion).

In exemplary embodiments, microbial survival and/or transfer potential (e.g., expressed in terms of microbial numbers or growth observed after transfer plating from the contaminated surface/material) from contaminated test samples (of either the fine particulate ion exchange polymer salt, or polymer composites made therewith) is less than 50% of microbial survival and/or transfer potential observed from control samples. In other embodiments, the microbial survival and/or transfer potential for test materials is less than 25%, 15%, 5%, 1%, or 0.0001% of the microbial survival and/or transfer potential observed from control materials. These and even higher levels of decontamination and transfer risk reduction are achieved for various microbial pathogens, including different forms of pathogenic bacteria, as well as pathogenic fungi and other microbial pathogens. A variety of standard decontamination and microbial transfer assay formats and protocols are well known and widely accepted in the art, which can be routinely practiced to enable characterization of the behavior of antimicrobial materials, articles and devices of the invention. In exemplary, antibacterial materials and composites, the level of bacterial, fungal and/or viral control and decontamination mediated by polymer salts and composites of the invention will correspond to at least a 50-75% reduction, often a 75%-95% reduction, up to a 95%-100% reduction and/or prevention of persistent contamination and/or transfer risk.

Results for post-contamination transfer potential, or infection risk, are even more surprising and beneficial using the antimicrobially active materials and composites of the invention. The subject materials and composites have such novel and powerful surface antimicrobial efficacy, they can substantially eliminate surface-to-living subject transfer of viable pathogens targeted by their surface-loaded ionic antimicrobial agents. For ease of description, retransmission potential (e.g., as measured by ability to transfer viable colony forming units of a targeted bacterium from a contaminated surface following a “decontamination period” (of, e.g., 10-30 minutes, 1-3 hours, or longer) is reduced by at least 75-95%, often greater than 95%, and reproducibly at levels of up 98-100% compared to similarly contaminated controls of like polymer materials that are not antimicrobially activated according to the invention.

The profound antimicrobial surface activity exhibited by novel polymer composites of the invention renders these materials widely effective against a large host of the most serious bacterial contaminants found in institutional care settings and environments. Effective materials and products are provided against the most refractory, costly and dangerous sources of infection found in medical and veterinary care hospitals, assisted living facilities, penal housing institutions, food processing and packaging facilities, and HVAC and other environmental control systems, among other environments as depicted in FIG. 2. Targeted microbes subject to reduction of surface contamination, and elimination of surface-to-live subject transfer risk, as described, include, for example, Staphylococcus spp. Pseudomonas spp. Escherichia coli, Klebsiella pneunioniae, Legionella pneumophila, Mycobacteria, Streptococcus spp, Acinetobacter, Haemnophilus, and Enterococcus, Aspergillus, and Listeria (including Monocytogenes sp. responsible for listeriosis). In addition, antimicrobial resistance has become a known threat to patients in hospitals, long term care facilities, and in our communities. Recently, a group of antibiotic resistant bacteria including Enterococcus faecalis, Staphylococcus aureus, Klebsiella pneumoniae, Acinetobacter baumanii, Pseudomonas aeruginosa, and Enterobacter spp., collectively dubbed ‘the ESKAPE pathogens’ i.e., those capable of ‘escaping’ antibiotics, and further communicating their evolving pathogenesis, and resistance genes, have raised concerns. The evolving threats of healthcare-associated infections (HAIs) are growing, threatening more lives every year.

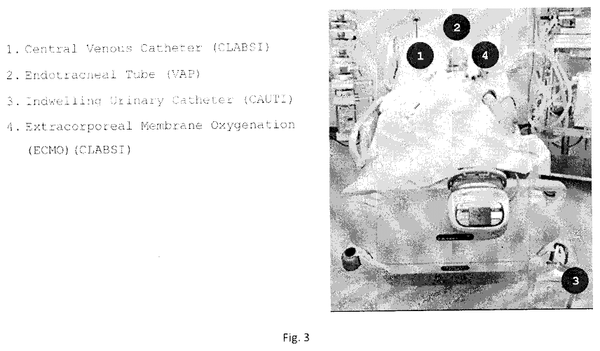

During the ongoing Covid-19 pandemic, some HAIs became confounding factors for the seriously ill patient. The reason for this is that patients with severe illness are dependent upon a number of lifesaving interventions. These can include a central venous catheter for medication delivery, an endotracheal tube for mechanical ventilation, an indwelling urinary catheter, and in the case of those patients at severe risk of dying, the use of extracorporeal membrane oxygenation (ECMO) as depicted in FIG. 3. All of these lifesaving devices are tubular in nature and reside inside and outside of the body. Each of these devices carries an independent set of risks. Central venous catheters and ECMO increase patient risks to central line-associated blood stream infections (CLABSI). CLABSIs increased by more than 500% according to surveillance during the pandemic. Mechanical ventilation raises the risk of ventilator-associated pneumonia (VAP) by 40-86%, significantly raising the risk of mortality. Catheter-associated urinary tract infections have increased by about 10% during the Covid-19 pandemic. As such it is difficult to know if this is a natural perturbation or an increase due to the increased use of indwelling catheters. FIG. 3 is representative of the Covid-19 patient with severe disease undergoing lifesaving device treatment.

Yet additional advantages afforded by the instant invention include a novel utility and efficacy against infectious targets resistant to many drugs, such as MRSA (methicillin-resistant Staphylococcus aureus), resistant Streptococcus strains, and resistant airborne pathogens such as Mycobacterium tuberculosis and Legionella pneumophila. Using the embodiments of the invention described herein, resistant organisms may be controlled or eliminated using compositions of distinctly modified fine particulate active ion exchange polymer salt materials in combination. In one embodiment a silver-modified fine particulate active ion exchange polymer salt material is combined with a chlorhexidine-modified a fine particulate active ion exchange polymer salt material and the mixture is added to a polymer composition to produce a binary delivery system. Because each of the antiseptics kills bacteria using unique mechanisms, the likelihood of resistant strains evolving is greatly diminished.

Yet additional advantages afforded by the instant invention include a novel utility and efficacy against infectious fungal diseases such as onychomycosis (fungal infection of the toe- and fingernails), tinea pedis, jock itch, ring worm, or cutaneous candidiasis. Antifungal agents can include copper (II), polyenes, imidazoles, triazoles, thiazoles, allylamines, echinocandins (caspofungin), flucytosine, and crystal violet. Generally, the aforementioned functional compound types may be used topically more effectively than by oral delivery. For example, for the treatment of onychomycosis, where Trichophyton rubrum is the most common dermatophyte involved in onychomycosis. Other dermatophytes that may be involved are Trichophyton interdigitale, Epidermophyton floccosum, T. violaceum, Microsporum gypseum, Trichophyton tonsurans, and Trichophyton soudanense. Topical agents include: clotrimazole, amorolfine or butenafine nail paints. All of these compounds are amenable to incorporation into the fine particulate organic ion exchange materials. Topical treatments need to be applied daily for prolonged periods, often up to a year or longer. In exemplary embodiments, a terbinafine-modified fine particulate active ion exchange polymer salt material will serve as an effective topical treatment (e.g., wherein this salt is incorporated into a hydrophilic lacquer and spread on the nail bed). In another embodiment illustrating the flexibility of the invention to mix and match active ion exchange species with diverse antimicrobial activity, a laboratory bench used for tissue culture is fabricated to include a mixture of antibacterial, antifungal, and antiviral agents, broadly reducing highly costly contamination of cell lines from environmental contamination.

In more detailed examples, Cu(II)-SCER or Cu(II)-WCER, or a combination thereof, is added to a lacquer, acrylic or other appropriate polymer, and the composite is applied to the nailbed for treatment. Similarly, Cu(II)-polystyrene sulfonate may be added to a lacquer containing isopropanol for painting the nailbed.

Antiviral compounds such as acyclovir (a synthetic nucleoside for treating herpes zoster and genital herpes), zidovudine or azidothymidine (a nucleoside analog for treating HIV/AIDS), abacavir (a nucleotide reverse transcriptase inhibitor), and lamivudine (a nucleoside nucleotide reverse transcriptase inhibitor) are readily bound to the fine particulate active ion exchange polymer to yield the organic ion exchange salt. One potential application for the antiviral-modified fine particulate active ion exchange polymer salts is to include the particulate into a hydrophilic matrix for placement into the vagina or anus for the delivery of the drug over time. Both of these locations are ideal for drug delivery due to the high vascularity thus allowing the drug to be effectively administered.

Other candidate active agents for the treatment of parasitic diseases can be incorporated onto the organic ion exchange backbones. For example, chloroquine, mefloquine, or doxycycline for the treatment of malaria can be readily bound to strong or weak cation exchange resins, as well as phosphates such as cellulose phosphate. Compounds for the treatment of Amoebozoa infections that cause dysentery including azoles (metronidazole and tinidazole), diiodohydroxyquinoline, and paromomycin for example can be employed with IRP69, IRP64, or polyphosphates. Helminth (nematode) infection particularly of the intestinal tract in humans and livestock can be treated using IRP69-, IRP64- or polyphosphate- organic ion exchange materials modified to include piperazine, benzamidazoles, levamisole, pyrantel, or morantel.

In an exemplary embodiment, a water filtration device fabricated from a non-woven fabric (e.g., polyester) filtration units formulated to include one or more of the antimicrobial additives of the present invention may be used in the sanitation of water. In another embodiment yet additional advantages afforded by the instant invention include the ability to yield antiparasitic-modified fine particulate active ion exchange polymer salt materials in order to provide novel utility and efficacy against infectious parasitic diseases that include treatment of sleeping sickness caused by Trypanosoma brucei) using Melarsoprol-modified material, sleeping sickness using Eflornithine modified material, vaginitis caused by Trichomonas using Metronidazole-modified material, intestinal infections caused by Giardia using Tinidazole-modified material, the treatment of visceral and cutaneous leishmaniasis using Miltefosine-modified material.

The novel biologically active polymer salts and polymer composites of the invention remain fully biologically active during preparation and for an extended period of shelf life thereafter, even though preparation of the polymer salts in fine particulate form involves non-solvent exposure and temperatures elevated to 85° C. or higher, and despite that curing of the polymer composites often involves elevated temperatures of up to 150° C., or 200° C. or higher. In addition, the biologically active polymer salts and polymer composites remain active with the biologically active ionic agent incorporated therein being stable to degradation, oxidation, chemical decomposition, and photodegradation for an extended shelf period after production as described. Additionally, the novel biologically active polymer composites of the invention retain not only their biological activity potential, but also their structural integrity for extended shelf and use periods. This activity retention and structural stability is marked by no greater than about 2 to about 5% of chemical loss, degradation, decomposition, destructive hydrolysis or oxidation for the biologically active ionic agents incorporated in the polymer salts and composites, and no greater than about 2 to about 5%, loss of tensile strength, environmental stress cracking, hardness change, or loss of elasticity of the composites during production, including during extended curing of composites at 200° C. with the exception of fine particulate organic ion exchange powder salt materials that may interfere with cure as a consequence of interference with a catalyst for example. In other embodiments, the stable retention of biological activity structural integrity of these novel polymer composites fabricated as compatible blends, i.e., the fine particulate organic ion exchange powder salt material does not interfere with curing of polymer systems or used as matrix materials, is marked by no greater than about 1 to about 5 wt. % loss under reasonable operating conditions and when tested alone, the resin systems exhibit remarkable stability well beyond the stability measured using the simple ion salt counterparts of the biologically active component. In general, the fine particulate organic ion exchange salt materials, in powder form, possess overall greater chemical stability, reduced thermal degradation and decomposition, and greater stability to destructive hydrolysis or oxidation for the biologically active ionic agents incorporated in the polymer salts and composites, and no greater than about 5 to about 15%, loss of tensile strength, change in hardness and/or modulus, or loss of elasticity for the composites over 1-3 months, 6 months, and up to a year or more in normal storage conditions (e.g., at standard laboratory room temperature and humidity, without use or mechanical wear). In more detailed embodiments, activity retention and structural stability is marked by no greater than about 1 to about 20% of chemical loss by exchange, degradation, decomposition, destructive hydrolysis or oxidation for the biologically active ionic agents incorporated in the polymer salts and composites, and no greater than about 1 to about 20%, loss of tensile strength, environmental stress cracking, hardness change, or loss of elasticity for the composites following extended exposure (up to 1-3 hours or longer) of the cured or hardened composites to extreme temperatures exceeding 200 degrees, 300 degrees and even 400° C. (allowing for a much broader array of clinical and industrial uses and post-production treatments of these novel composites and biomaterials).

After periods of use, the surfaces of biologically active composites and related biomaterials of the invention may start to lose their peak biological activity potential. For example, the biologically active ionic agents incorporated in the composites may become partially exhausted due to mechanical abrasion and other mechanisms of loss, ionic dissociation (particularly when used in contact with physiological or other ionic fluids), chemical reaction, chemical change by oxidation or hydrolysis, photodegradation, or other types of removing, discharging, destructive, transforming or deactivating factors.

In alternative embodiments, the biologically active ion exchange resin material is integrated within an outer and/or inner surface portion only of a solid polymer structure or a composition of materials such as with a coextrusion and may be absent from all or part of deeper internal, core or interstitial portions of the polymer structure. In other alternative embodiments, the biologically active ion exchange resin material is integrated within a coating or multi-layer laminate formed of the solid polymer, which can be applied or co-formed to cover a different polymer or non-polymer structure that does not incorporate the biologically active ion exchange resin material or can incorporate a reduced concentration so as to prevent undesirable tissue reactions or toxicity. In an exemplary embodiment, a 6.0 Fr ureteral stent comprising an Ag-SCER silicone rubber composite is coextruded so that the outer “tissue contact layer has a concentration of 1.5 wt % Ag-SCER and the inner layer has a concentration of 2.0 wt % or higher. The higher concentration of the inner lumen can better prevent microorganism proliferation and the outer layer at 1.5 wt % is more tissue compatible as demonstrated by ISO 10993 testing to include a 26-week implant study that demonstrated the material to be well tolerated.

Within these and related embodiments, as the biologically active ion exchange resin material, and or the integrated ionic biologically active agent, is discharged, degraded, dissociated or exhausted at the surface of the active polymer composite (e.g., by mechanical wear or debridement, light or chemical degradation, chemical reaction on contact with external chemical species, oxidation, hydrolysis, decomposition, and/or ionic dissociation of the active ionic agent through exposure to physiological or other ionic fluids, chemical reaction), most or substantially all of an original surface biological activity of the polymer structure is maintained, either passively, for example by “erosive recharging” (wearing that debrides old surfaces and brings out a newly-exposed, fully charged surface), or actively through manual recharging (e.g., manual debridement to expose a new surface with full activity potential, such as by abrasive polishing/burnishing), or chemical recharging or reconditioning.

In one “self-regenerating” embodiment of the invention, recharging of surface biological activity following partial or complete “discharge” of the ionic biologically active agent initially present (e.g., after the polymer composite is newly formed and hardened) at the polymer surface is achieved by passive erosive recharging. Here, normal contact abrasion (e.g., rubbing of surgical or catheter tubing against another object) wears away an immediate, outermost surface “layer.” This abrades away or debrides the outermost layer, which will frequently comprise some discharged material (i.e., where the outermost layer of polymer exhibits less than the initial loading or activity capacity of the biologically active ionic agent). This exposes a “new” “regenerated” or “restored” outer layer fully invested with the ionic biologically active agent in a non-degraded or discharged state. In exemplary, “self-disinfecting” embodiments, products incorporating antimicrobially-active polymer composites of the invention may have an erodible surface and function such that abrasion of the erodible surface exposes new (originally subsurface) antimicrobial particles incorporated within the composite matrix (active fine particles of ion exchange resin material incorporating an ionic antimicrobial agent).

Stable composite materials of the invention may be restored to full activity following, for example, natural or mechanical wearing away of the surface, such as by abrasion or burnishing. This restorable functionality is particularly useful for antimicrobial materials used in hospital settings, where patient infections often arise by contact with or indirect exposure to contaminated fomite surfaces. For this use, antimicrobial composites of the invention provide for increased regenerative activity proportionate to the degree of contact/wear. In other embodiments, restoration/regeneration of activity is provided by deliberate manual abrasion or polishing of a subject surface to remove an exhausted outer portion of the material, wherein the active agent is distributed not only at the surface, but within subsurface layers of the polymer, or throughout the polymer composition, which layers or material is exposed by abrasion or polishing. Abrading and polishing can be done by any number of materials such as abrasive sheets, abrasive pastes, and abrasive gels. Such abrasive and polishing materials may contain different grades of abrasive material with the finest necessary grade leaving the outer surface smooth so that there are no contaminable pores or voids. In one exemplary embodiment, a port of a central venous catheter (CVC) comprising a polycarbonate (female) luer connector fitted with a silicone rubber septum and both components formulated to include fine particulate organic ion exchange powder salt in Fe(II) or Cu(II) form and at the time of pairing the female luer connector of the CVC with the male luer counterpart for the delivery of medicament or nutrition, the female portion of a luer connector is swabbed with a sponge containing hydrogen peroxide solution. The sponge may be fitted onto a male luer connector in order to allow the cap to be turned to rub/swab the hydrogen peroxide moistened sponge across the surfaces of the female luer connector thus enhancing fluid contact and the uniform generation of superoxide as a means of adequately disinfecting the inner surfaces of the connector. This embodiment is described a means of preventing catheter-related blood stream infections (CRBSIs).

Various assays and model systems can be readily employed to demonstrate the effectiveness of copolymeric and other ion exchange materials for integrating therapeutically useful counter-ions within polymer composites of the invention. For example, antimicrobial effectiveness may be demonstrated by Kirby-Bauer Assays. The Kirby-Bauer Assay (Disk diffusion/Zone of inhibition) is a test method that uses antimicrobial-impregnated wafers to assess whether particular bacteria are susceptible to specific antimicrobial agents. In this method, bacteria are grown on agar plates in the presence of samples containing relevant antibiotic agents. If the bacteria are susceptible to a particular antibiotic, an area of clearing surrounds the sample where bacteria are not capable of growing (referred to as a zone of inhibition).

Kirby-Bauer assays can be used to evaluate the effectiveness of the materials (ion exchange material loaded with oligodynamic metal ions and ammonium ions, and blended silicone LSR materials) and the materials can be shown to possess broad antimicrobial capability against Gram-negative and Gram-positive organisms, and fungi including but not limited to: Staphylococcus, Pseudomonas, Escherichia coli, Klebsiella pneumoniae, Legionella, Mycobacteria, Streptococcus, Acinetobacter, Haemophilus, and Enterococcus. As well as Aspergillis. These agents can be tailored to address multidrug resistant organisms and a variety of airborne pathogens including Mycobacterium tuberculosis and Legionella pneumophila.

Efficacy may additionally be more rigorously demonstrated through the use of ISO 22196. ISO 22196, Measurement of antibacterial activity on plastics and other non-porous surfaces, has been utilized for the evaluation of antimicrobial ion-modified resins incorporated into a variety of materials. Exemplary antimicrobial ion exchange modified materials have demonstrated between 3-log to 7-log overall reductions in bacterial organism counts for a diverse array of species, including Escherichia coli and Staphylococcus aureus, at as little as 1.0 wt. % loading levels (% by weight of active fine particulate polymer salt per final composite weight, determined prior to mixing of polymer salt with thermoplastic or thermoset polymer).

Selection of optimal polymer loading with active ionic agents for composites and coatings of the invention will depend upon the composition and intended use of the subject polymer and active agent(s). Organometallic strong cation exchange resins (SCERs) of Ag, Cu, Li. Zn etc., are readily prepared, for example, by reaction of metal acetate salt and sulfonic acid forms of a polymer. In some instances, the acid form is available from the manufacturer. Otherwise, the acid form can be synthesized by treatment of the sodium salt form with hydrochloric acid (HCl) to restore the sulfonic acid form from the neutral salt form. Acid-base titration of the acid form of the resin provides the resin exchange capacity (Eq/L), an important gauge of the loading potential of the resin that enables the determination of how much metal acetate is needed to convert the resin to its metal sulfonate form. Following the sulfonic acid+metal acetate reaction, the metal functionalized resin is purified and dried and the resin analyzed for elemental metal content, either by inductively coupled plasma optical emission spectroscopy (ICP-OES), atomic absorption (AA), or metal titration. These results (ICP-OES vs. titration) have been shown to correlate reliably for the group of SCERs that have been converted to their metal salt counterparts, for example with Ag-SCERs generally aggregating at 34-36 wt % Ag+ and copper derivatives generally aggregating at 12-13 wt % Cu(II). An illustrative Ag-SCER batch MV_03-118 demonstrated a wt % of Ag of 34.9% by ICP-OES and 34.5% by titration. Thus, loading of this particular resin composition into polyethylene at 1.0 wt % (resin to final polyethylene resin composition), for example, equates to about 0.345-0.349 wt % Ag in the final composite. In another example, the loading of Cu(II)-SCER into an acrylic latex enamel paint at 3.0 wt % (dry paint solids basis) equates to a paint composition approximating 0.36-0.39 wt % Cu(II) loading for the finished dry/cured coating. Generally, the useful loading levels for the various resin derivatives have been shown to be from 0.5-5.0 wt % loading of the resin. For the silver derivatives, this corresponds to a range of Ag+(wt %) approximating 0.17-1.7. For Cu(II), this approximates a range of 0.06-0.6 wt %. The batch-to-batch reproducibility of antimicrobial ion loading is dependable, as determined by repeated elemental analyses across batch production. This has importantly allowed for standardizing of manufacturing methodology at both bench and full production scale.

Efficacy of the biomaterials provided herein may be demonstrated, for example, through the use of the more rigorous ASTM E2180 method (ASTM International, West Conshohocken, P A, 2007). ASTM E2180 is a method whereby treated test samples are inoculated with the test organism mixed within a semi-solid agar “slurry” to facilitate surface interaction. The test organism is thus exposed for attachment/colonization on the surface of the test material typically for 24 hours. Control samples of the same material that are not “active” according to the invention (e.g., a silicone polymer that does not contain active fine particulate polymer salt material) is similarly inoculated and evaluated. The test and control samples are then treated with a neutralizing solution comprising tryptic soy broth (base), lecithin (1.0 gram/liter) and Tween 80 (7.0 grams/liter). With this solution, cationic antimicrobial agents are neutralized in order to prevent them from continuing to eliminate bacteria during the test procedure, the surfaces are subsequently washed and samples are quantitatively assayed for antimicrobial activity (e.g., bactericidal and/or bacteriostatic activity). The resulting plates are incubated, and the number of survivors can be enumerated by direct surviving cell counts and/or by determining both survival and viability for reproduction through subsequent detection of colony production (colony forming units or CFUs). This provides for measurement and expression of “decontamination efficacy” of the novel biomaterials of the invention, which may be expressed as a percent reduction of viable microbes capable of surviving and/or reproducing. These values are determined for both test and control materials, and on this basis relative efficacy values for “decontamination activity”, “bactericidal and/or bacteriostatic activity”, and “transfer risk reduction”, among other measures of efficacy, can be determined. Comparable assays are routinely implemented to determine antifungal (fungicidal and fungistatic) activity, antiviral activity, and antiprotozoal (e.g., amoebicidal) activity.

Common test organisms utilized in methods for determining antibacterial activity include Escherichia coli and Klebsiella pneumoniae. Exemplary antimicrobial polymer composites of the invention have been evaluated and shown to affect 3.69 and 3.72 log reductions against these bacteria, respectively. In other exemplary embodiments, antimicrobial polymer composites having as low as 1.0 wt. % loading of the composite with fine particulate active ion exchange polymer salt have been tested and shown to affect _{[DV2]}6.2 and 5.98 log reductions in these respective organisms at as little as 1.0 wt. % loading. The data from these and other assays demonstrate the ability of active ion exchange polymer salts and polymer composites incorporating these novel materials as potent drug delivery and surface-active biomaterials for use in clinical, industrial and other applications.

In certain aspects of the invention, the biological activity potential of active polymer composites can be varied by selecting different effective loading amounts particle distributions within composites for the active, fine particulate ion exchange polymer salt or the concentration of a soluble linear sulfonated polymer salt such as polystyrene sulfonic acid benzalkonium salt. Other examples include polystyrene sulfonic acid cetylpyridinium salt. The former compound (polystyrene sulfonic acid benzalkonium salt) is soluble in alcohol and nearly insoluble in water and the latter (polystyrene sulfonic acid cetylpyridinium salt) is highly soluble in water and alcohol. Either of these may be applied to surfaces to include solid countertops, touch surfaces such as bed rails, personal protective equipment, such as scrubs lab coats, N95 masks, hepa filters and other melt blown fabrics, non-woven air filters, and other surfaces where a dilute polymer salt can provide protection from bacteria, fungi and viruses that may be deposited onto a surface.

Activated polymer composites of the invention can be formed as flexible or rigid biomaterials in virtually any shape, size, thickness or structural relationship with other materials (e.g., Teflon, nylon PTFE, stainless steel, titanium, etc.) to make biomedical articles, tools and devices through the use of molding, extrusion, or coating by spraying, dipping, painting, or electrostatic or fluidized bed deposition with subsequent curing. The polymer composites may incorporate into biomaterials, textiles and articles of manufacture, for example, by casting, molding or assembling the composites directly into an article of manufacture, coating or laminating the composites over articles of manufacture, or mixing the composites with textiles or other precursors of articles of manufacture, among other fabrication modes and formulae.

The invention thus provides a valuable assemblage of biologically active polymer composites for construction of clinical, therapeutic and diagnostic materials and devices. Operative embodiments employ the biologically active polymer composites of the invention incorporated within such diverse materials and devices as antimicrobial disposable blotters, sponges, and surgical wear (e.g., gloves and shoe covers), permanent or temporary coverings for traditional fomite surfaces such as surgical trays, operation room (OR) equipment, drug and fluid delivery devices, catheters and tubing, cardiovascular and orthopedic implants, stents, grafts, and anchoring or suturing materials and devices (e.g., pins, posts, staples, and sutures) and a diverse array of comparable laboratory equipment (e.g., materials, components, tools, containers, disposable and non-disposable coverings and textiles for use in forensic, diagnostic, microbiological and tissue culture laboratories).

Additional biomaterials, components, coatings, devices, furnishings and equipment in which the novel active polymers of the invention are beneficially incorporated include, for example, food-processing equipment, packaging and products; consumer clothing and apparel; first responder protective wear and gear; athletic (e.g., sports therapy and gymnasium) materials, equipment and clothing; lavatory materials, furnishings and equipment, transportation equipment (e.g., high-contact/heavy use surfaces on buses, subways, trains, planes, cruise ships), and HVAC and other air and fluid circulation and management systems and components (e.g., coatings on air ducts, connectors, ports, collectors, fan blades and housings, impellers and filters).

Exemplary medical, laboratory and industrial materials and devices of the invention

include active polymer composites integrated within paints, floor coverings and coatings, wall materials, joining and adhesive compounds for medical devices, walls and furnishings, countertops, laminate materials, filters, and appliances. These exemplary materials may be formulated as solvent-based lacquers, water-based latexes, epoxies or polyurethanes, or as two-part curing systems that can include epoxies and polyurethanes to include foams. In one detailed example, a highly-durable surface coating/paint for vehicles, storage containers, aircraft, watercraft, or other comparable painted equipment are formulated to include Cu(II) or Fe(IL)-SCER. These coatings facilitate decontamination of surfaces, for example following a Chem/Bio warfare attack. While remaining in the field, the equipment is first treated with a salt solution of at least 20 millimolar, i.e., 0.020 moles (1.17 grams) of NaCl (58.45 grams/mole) and subsequently the surface is treated with hydrogen peroxide solution (≥3.0 wt. %). The sodium ion facilitates liberation of Cu(II) and the peroxide acts in conjunction with Cu(II) to form hydroxyl radical. The Fenton reagent has been utilized extensively for remediation of polluted soil for example, particularly using Iron (II). These coatings and materials are also useful in vehicle paints (epoxy-based) and may be employed for protective gear, air and water filters and the like. In one exemplary embodiment, strong cation exchange resin is converted to the Cu(II) salt form using resin or approximately 3 mm or larger. The converted resin can be loaded into filter packs (columns) and used in-line and in series as needed in water systems to remove bacteria that are present in the source. Microorganisms that may be found in water includes Coliforms, E. coli, Shigella, Vibrio, Salmonella, Legionella, and Enterococci. Enteroviruses that can be found in water include polioviruses, echoviruses, coxsackieviruses. Other viruses that can be found in water also include Norwalk virus and Rotaviruses. Protozoans and Parasites found in water include Entamoeba, Giardia, and Cryptosporidium. Bacteria that become adherent to the resin will be killed with contact. Viruses can be inactivated by surface contact or local concentrations of Cu(II). As long as the eluent from the columns does not yield water with copper levels exceeding the safe limit for drinking water of 1.3 mg/liter (EPA), the water can be consumed. The amount of Cu(II) released will be dictated by the ion content of the source water. This novel concept of sterilizing water can be beneficial to space vehicles, ships, submarines and the like where water systems are contained and must be able to prevent biofilm deposition onto the surfaces of the system. In exemplary embodiments a columnar filter (FIG. 1) is loaded with a Cu(II) derivatized weak cation exchange resin (WCER), Cu(II)-WCER (which releases Cu(II) at a much lower rate), or with both Cu(II)-SCER and Cu(II)-WCER(II), for water and other fluid contamination effective against a wide range of microbes, viruses and protozoan contaminant organisms.

In another exemplary embodiment, hospital rooms, cruise ship areas, prison units and other locations with surfaces/facilities at risk of colonization by infectious organisms, can be constructed or modified using antimicrobial paints or coatings of the invention (e.g., to provide antimicrobial painted walls, epoxy flooring, powder coated fixtures, etc.) incorporating Cu(II) or Fe(II) SCER or other biologically active additives of the invention. A machine that can vaporize 35% hydrogen peroxide and a minimal ion containing water solution or plain tap water vapor can broadly activate the subject surfaces to generate hydroxyl radical and eradicate even persistent organisms including Candida auris, also known as C. auris, a drug resistant organism that has demonstrated persistence against hydrogen peroxide vapor alone. Surfaces in the spaces described (hospital rooms and cruise ships etc.) may be disinfected using a temporary surface deposited from a solution of a linear polymer such as a polystyrene sulfonic acid quaternary ammonium salt (QAC-PSS). The quaternary ammonium ion may be one of cetylpyridinium, benzethonium, didecyldimethylammonium, or benzalkonium for example.

The invention further provides a wide array of novel compositions, products and devices useful in agriculture. In one illustrative embodiment, a dilute polystyrene sulfonic acid copper salt (Cu(II)-PSS) composition is sprayed onto trunks, branches, buds or fruits of agricultural trees, effectively preventing pathogenic fungi and viruses from destroying or reducing crops, for example stone fruit crops. The use of Cu(II)-PSS in combination with didecyldimethylammonium (DDAC)-PSS in related agricultural embodiments will yield a synergistic antimicrobial effect against harmful bacteria, fungi and viruses, based on complementary antimicrobial activity between QACs and copper +2 ion. This discovery of synergy between Cu(II) and QACs provides a novel means within these and other aspects of the invention for biofilm disinfection. Ammoniacal copper quaternary (ACQ) is a combination of copper oxide (CuO) with the QAC didecyldimethylammonium chloride (DDAC)2 that has been used as a fungicidal and insecticidal wood preservative since the early 1990s. ACQ and other Cu-QAC combinations in polymer composites of the invention can also be applied successfully to treat biofilms in a wide range of other environments, where surface-associated microbial growth is unwanted or damaging. The novel combination using PSS as a coating with these active agents renders the desired activity persistent for longer-term delivery, for example in agricultural settings where the coated objects are subject to rain, wind and insect activity. The resulting materials and methods provides for increased crop yields with reduced environmental contamination.

In yet another exemplary embodiment, ceiling tiles and related architectural panel materials can be coated with a polymer incorporating the Cu(II)-SCER, for example applied as a surface paint containing Cu(II)-SCER, and this coating can be activated with tap water, saline or saline with dilute hydrogen peroxide to address infectious pathogens (e.g., Candida auris) colonizing pores of the material. Tiles and other articles can similarly be coated with polystyrene sulfonic acid salts of quaternary ammonium ions and metal ions including zinc (II), or copper (I) or copper (II).

Exemplary medical and laboratory devices and equipment that can be partially or completely constructed of the novel biomaterials provided here include drug and fluid delivery and catheter tubing, molded components, coatings, surgical tools and equipment, biohazard disposal surfaces and containers, hospital bedding, gurneys, stretchers, textiles including surgical scrubs, gowns, surgical drapes, bedding, wound dressings, etc. Other, similar assemblages of materials, devices and applications are contemplated for food harvesting, handling, processing and serviced industrial tools, textiles and equipment, and for heating, ventilation, and air conditioning (HVAC) system components including filters, heat exchangers, coils, duct work, fans, humidity control components, heat pumps, vents, manifolds, water drains and P-traps constructed from non-plasticized polyvinylchloride (PVC) and the like. Yet additional materials, devices and applications will incorporate the active polymer composites of the invention within bulk storage containers, public transportation surfaces, office equipment, food conveyers, clean rooms, consumer products (children's toys, highchairs, bathroom cleaning appliances, sexual aids, hygiene implements such as toothbrushes, dental floss and skin and eye care materials, vaporizers and other devices).

Exemplary medical and hygiene products that will beneficially incorporate biologically active polymer composites of the invention include, for example, catheters, tracheostomy tubes, wound drainage devices/catheters, stent, implants, introducers, stylets, sutures, shunts, gloves (latex, neoprene, viton), condoms (polyurethane, latex, silicone), contact lenses, gastrostomy tubes, cardiovascular stents, prostheses, pacemaker and ICD pulse generators, grafts, valves and implants, surgical guidewires, urine collection devices including drainage bags, medical tubing, intravenous catheters, urinary catheters, Foley catheters, vascular access and dialysis catheters, peritoneal dialysis catheters, pacemaker leads, urological catheters, wound dressings, medical sheeting, endotracheal tubes, tracheostomy tubes, septa used for piercing with needles for sterile retrieval of drugs from supply vials, or for delivery of drugs, nutrients, saline or other materials via intravenous connectors, clamps, shunts, catheter ports, hubs, catheter port cleaning cap devices (for ensuring that septum and port are sterile for the providing drug therapy, nutrition, or removing body fluid), surgical repair constructs and meshes, and many other materials and devices.

Exemplary wound dressings of the invention include calendared silicone sheet composites incorporating active, fine particulate ion exchange polymer salts, such as a silver ion exchange salt. The composite calendared sheet may be attached to a release liner, and the sheet may be fenestrated to allow transmission of water from a wound bed when the dressing is in contact with wound tissue. These and related dressings are particularly effective for dressing burns, as the silicone is non-adherent and the presence of the active, fine particulate ion exchange polymer salt precludes the attachment and survival of bacteria and fungi.

Exemplary polymer film-based devices of the invention include plasticized PVC urine drainage bags comprised of PVC composite incorporating approximately 0.50 wt. % Ag-SCER. Drainage bags are known to be a source of infection for patients where the reflux of urine back into the bladder leads to infection. Although Cu(II)-SCER in PVC has been shown to be very effective against a broad array of pathogens, it was demonstrated that over a period of 21-days, the Cu(II)-SCER composite was rendered ineffective with exposure to urine whereas the Ag-SCER PVC composite remained broadly effective for 28-days.

Among significant industrial and public utilities uses, the biologically active polymer composites of the invention are particularly well adapted for useful integration in air and water-handling systems, including heating, vacuum, and air conditioning (HVAC) components, conduits, fittings, filters, recirculators, pumps and the like. The heating, vacuum, or air conditioning components can include one or more of duct work, heat exchange coils, heat exchangers, fan components, vents, energy-recovery ventilators, blower components, ballasts, levers, air filters, water filters, heat pumps, fluid handling systems and/or the like. Coatings incorporating organic ion exchange polymer salt materials embedded within the composite (or composite surface layer(s)) require activation by a salt with which exchange can occur. As such, in environments where the moisture present is condensate of atmospheric humidity, ion concentration may be low or non-existent. Thus, for situations where this is possible, the coatings can incorporate a sacrificial salt such as an alkali salt of a dioic acid, an aluminum phosphonate, or a non-corrosive salt such as sodium gluconate.

In other embodiments, the biologically active polymer composites of the invention are uniquely adapted for improving safety and performance of building, flooring and surface construction materials, including hospital, laboratory and home building, construction and sealing and adhesive materials. Among such materials that will beneficially incorporate surface paints or coatings of these active polymer composites are flooring materials, countertop materials, wall construction materials, caulking compounds, foaming insulation materials, and cast or molded enclosures for bathroom applications. Exemplary uses for these novel materials include prevention and elimination of toxic molds (e.g., Stachybotrys chartarum) and other dangerous or destructive species of fungi, such as Alternaria, Aspergillus, Aureobasidium, Chaetomium. Cladosporium, Fusarium, Mucor, Candida auris, Trichoderma, Ulocladium, and Penicillin, that can take up residence in homes, hospitals, extended care facilities, disaster relief housing or barracks, as well as a range of other structures and environments susceptible to colonization and proliferation of toxic bacteria, fungi, viruses or other unwanted microorganisms. In general, the polymers and coatings described herein are integrated within or coated onto materials, for example building materials, such as gypsum drywall, whereby the polymer composites integrating antibacterial, antiviral, and/or antifungal active ionic agents, such as Cu(II)-SCER into the coating material/paint, or by the use of water-soluble Cu(II)-polystyrene sulfonate deposited onto the surface from a dilute alcohol solution, for example. Whereas gypsum drywall producers have attempted to address these problems with mold and mildew by using fiberglass in place of paper, the primers and other coatings provided herein, for example employing Cu(II)-SCER, are less costly, easier to produce and more effective. In illustrative embodiments, a gallon of primer to coat 300 square feet of surface, comprising an active additive that adds a cost of $0.75/gallon to base primer cost, for an estimated total cost of $8.75/gallon, this gallon will cover roughly 4.7 sheets of gypsum drywall. This equates to approximately $1.86/sheet. At the retail level, fiberglass laminated gypsum drywall sells for about $3.70/sheet more than paper coated gypsum board. As the cost of the primer is reduced, the price per sheet is reduced. Other benefits for this solution may be obvious.

In other embodiments of the invention, the polymer composites of the invention can be used to construct finished fabrics derived from naturally occurring fibers or man-made materials, or from plant-based materials such as paper. The fabric materials can be constructed from one or more of a weave, knit, knot, crochet, or melt spun or unwoven (non-woven fabrics) and the antimicrobial additives of the present invention can be incorporated by inclusion into the fibers of manmade material prior to fabrication of yarn, thread or the like or the antimicrobial additives of the present invention may be added as a coating (sizing) onto the fabric. The textiles as described herein may be utilized to fabricate any variety of textile-based products to include clothing and garments such as shirts, socks and stockings, and pants that may find applications for example in sportswear, military, and hospital applications. Garments for use in hospital and healthcare environments may include surgical scrubs, neckties, and lab coats, as well as hospital gowns, pajamas, and undergarments for example. Other textile-based articles can include surgical masks, booties, and protective suiting for application in and around infectious diseases.

Synthetic fibers that can be spun at or below 250° C. are exemplary materials for inclusion of modified SCERs of the invention. Cu(II), Ag, and Benzalkonium (BA), cetylpyridinium (CP) or other quaternary ammonium modified resins can be incorporated into various fibers whether for weaving fabrics or the production of non-woven materials. In certain embodiments, silicone composite comprising Ag-SCER (0.5-3.0 wt. %) is fabricated into a polyurethane (or other appropriate polymer) sizing solution that can be applied to fabrics to protect against bacterial and fungal growth, formulated directly into the gel portion of a prosthetic sock or can be fabricated directly into a liner for use with the prosthetic_{[DV3]}.

In another exemplary embodiment, sodium polystyrene sulfonate is deposited onto a non-woven textile for use as a personal protective equipment garment, mask, surgical drape, hood, booties or the like. The polystyrene sulfonate coating is advantageous as it is capable of immobilizing aerosolized droplets that contact the surface by absorbing the water into the coating. Furthermore, sodium polystyrene sulfonate is a polyanion capable of inactivating enveloped viruses. As such, this coating is expected to be effective against SARS-COV-2. Pairing the PSS with an antimicrobial cation such as Cu(II), benzalkonium, or other quaternary ammonium ion is expected to provide enhanced protection. Cu(II) as bound to crosslinked versions of PSS has been shown to inactivate enveloped and non-enveloped viruses. The polymer backbone also minimizes any likelihood of toxicity.

In other embodiments, the self-disinfecting compositions may be used to make touch surfaces for use in one of a clinic, hospital, nursing home, long-term care facility, gymnasium, sporting facility, workout facility, kitchen, bathroom, recreation center, academic institution, cafeteria, watercraft, motorized vehicle, and/or disposal container. Touch surfaces as related to gymnasiums, recreation centers, and sporting institutions can include for example grips related to equipment and exercise machines, mats for warming-up and stretching, martial arts, boxing, and wrestling.

In exemplary embodiments of anti-fouling coatings provided herein, active fine particulate polymer salts of the invention can be incorporated into surface materials such as Dupont's Corian, an acrylic polymer containing alumina trihydrate. The use of a strong or weak cation exchange resins with Cu(II) modification are contemplated to provide surfaces that are at least as antimicrobial as copper metal surfaces. In order to facilitate disinfection of such a surface, a light misting of the surface with a saline solution will liberate enough Cu(II) to facilitate the killing of bacterial and fungal organisms. In another embodiment, epoxy can be substituted for acrylic to provide a tough composite material that can have similar applications.

In other exemplary embodiments the active fine particulate polymer salts may be incorporated into paints that can be applied inside clinical institutions, particularly in rooms that house patients with drug-resistant infections (e.g., burn units). In particular, the addition of Cu(II)-SCER is demonstrated to be highly effective when formulated into acrylic latex enamel paint at a concentration of roughly 1.5-3.0 wt %. The resulting coatings are easy to tint at this low concentration and these coatings are remarkably stable, resilient, and durable. For example, a painted panel (3.0 wt % Cu(II)-SCER, 5.0 micron particle size, acrylic latex enamel) was washed 3-times/week for a duration of 3-weeks using Lysol Institutional Cleaner (quaternary ammonium), 10% bleach, and LpH®se (acid phenolic cleaner and detergent) at the recommended dilutions. After each washing, the wash water (including that in the sponge) was collected and the wash water was measured for copper content using test strips (Hach 2745125) sensitive to 0.2 ppm 20 micrograms/liter). A self-imposed limit of 1.3 PPM (the safe limit for drinking water established by EPA) was never achieved in any of the wash eluent collected after each washing. This indicates that Cu(II) is not readily liberated even in the presence of disinfection solutions that contain cations such as Lysol (Industrial Strength Quaternary Ammonium Cation cleaner) or bleach (Na⁺OCl⁻). Similarly, the coatings can be applied on cruise ships where norovirus outbreaks are common. In the event of an outbreak, the use of a tap water or dilute salt solution over any affected area will liberate Cu(II). With the addition of hydrogen peroxide (H₂O₂), hydroxyl radical (HO—) can be liberated at, or near, the surface to further inactivate microorganisms or biofilm. Table 4 in the presented Examples provides a tabulated overview of the efficacy of several architectural acrylic latex enamel coatings to include Cu(II)-SCER (2.5 and 3.0 wt. %) and Cu(II)-SCER+Benzalkonium (BA)-SCER at 1.0 wt %+1.0 wt %. The duration of the assay varies from 2.0 hours to 0.5 hours. EPA has provided an antibacterial label to Paint Shield® (Sherwin Williams) for its ability to provide greater than or equal to 3.0 log reductions against Staphylococcus aureus, Escherichia coli, and Enterococcus faecalis within 2-hours. The data presented here was developed using the identical EPA method. These data demonstrate better and broader efficacy than Paint Shield®. In addition, Paint Shield® is not active against Gram negative organisms, aside from Escherichia coli. The Cu(II)-SCER acrylic latex enamel formulation described here demonstrated multi-log reductions against MRSA (gram positive) and Enterococcus faecalis (gram positive), Escherichia coli (gram negative), Acinetobacter baumanii (gram negative), Pseudomonas aeruginosa (gram negative), and Klebsiella pneumoniae (gram negative) in ¼ of the time (30-minutes) as reported for Sherwin Williams' Paint Shield© as shown in Table 4, below.

In other examples, the combination of antimicrobial ion exchange resins into materials can be used in prosthetics socks, stump shrinkers, and prosthetic liners for amputees, can minimize the presence of Staphylococcus (epidermidis and aureus) and Propionibacteria, all microbes of normal human flora. In addition, these composites can prevent Bacillus subtilis and fungal organisms known to cause athlete's foot that include Epidermophylon, Trichophyton, and Microsporum.

In yet another example, the combination of antimicrobial ion exchange resin, such as the silver form of a strong cation exchange resin (Ag-SCER), into silicone can be calendared, or extruded, into a sheet, fenestrated to allow the transmission of wound fluid, cut to size, packaged and sterilized by ETO or other appropriate sterilization method. The resulting wound dressing acts as a contact layer for burn or other wound management and the presence of Ag-SCER prevents biofilm from accumulating.

Similarly, socks and sheaths are also provided with gel attached to, or sandwiched between, the fabrics. The gel is usually made of silicone and provides excellent cushioning, pressure distribution and reduced friction. The thickness and stiffness of the gel will dictate the cushioning qualities of the sock. Because gel tends to flow from areas of high pressure to areas of lower pressure, maintenance of a more even pressure distribution is possible. If the sock or sheath is constructed with the gel exposed, the gel should be worn against the skin. This will help protect the skin from the friction forces created during walking, since the motion will tend to occur between the gel sheath and the prosthesis rather than between the gel and the skin. If the gel is formulated with a small percentage of Ag-SCER (0.5-1.0 wt. %) bacterial growth is deterred.

In certain embodiments of the invention, antimicrobial ion exchange resin powders can be incorporated into powder coatings for the purposes of application to a variety of metallic surfaces, such as appliances, aluminum extrusions, medium density fiberboard, for example by use of thermoset or thermoplastic powder coating. The thermosetting variety incorporates a cross-linker into the formulation. When the powder is baked, it reacts with other chemical groups in the powder to polymerize, improving the performance properties. The thermoplastic variety does not undergo any additional actions during the baking process as it flows to form the final coating.

Powder coatings (powder paints) are finely divided polymer compositions in the form of dry, solventless powders that flow into uniform smooth coatings and cure with heat, usually onto metal surfaces. Powder paints are commonly applied by electrostatic spray or fluidized bed. Powder paints are environmentally friendly, uniform, and durable and comprising paint particles of ˜20-70 microns in diameter with the key ingredients of the system encapsulated into the particles. Key components include: 1. A binder system (e.g., a resin) and a crosslinker for a thermosetting system; 2. Pigments and fillers; and 3. Additives that assist air escape, flow, accelerate cure, or provide antimicrobial character to coatings that are beneficially thicker and more durable than coatings made by brushing or spraying. Any component added to the powder paint must not inhibit cure or reduce the integrity of the powder system.

Antimicrobial coatings are important in today's powder coating market and silver technology is the main contributor and these powder coatings do not rapidly disinfect, disillusioning some industry insiders with its use. Although silver has a track record for coating application uses, it has limitations including cost, slow activity, and discoloration. Ionic copper is less expensive, faster to act, and does not discolor. The most common polymers used in powder coatings are polyesters, polyurethanes, polyester-epoxy (known as hybrid), straight epoxy (fusion bonded epoxy) and acrylics. Generally, the production of powders used for coating in this fashion are accomplished by mixing the polymer granules with hardener, pigments and other powder ingredients in an industrial mixer, such as a turbomixer, the mixture is heated in an extruder, the extruded mixture is rolled flat, cooled and broken into small chips, and the chips are milled and sieved to make a fine powder. This process is conducive with the formation of composites using fine particulate biologically active ion exchange polymer salt materials, to manufacture a full array of antimicrobially-coated materials, products and devices as described herein.

In more detailed aspects of the invention, powder coatings and powder paints provided herein are finely divided polymer compositions in the form of dry, solventless powders that flow into uniform smooth coatings and cure with heat, usually onto metal surfaces. Powder paints are environmentally friendly, uniform, and durable and are comprised of paint particles of ˜20-70 microns in diameter with the key ingredients of the system encapsulated into the particles as shown.

To formulate the powder coatings with antimicrobial additives according to the invention, the additive and powder are first premixed (e.g., in a vertical head mixer) and a homogeneous dry blend is subsequently run through an extruder at a melt temperature that allows homogeneity to be achieved (below temperatures that will induce substantial crosslinking). Once the material exits the extruder, the mixture is rapidly cooled, the blend is broken into flakes, and the flaked blend is subsequently milled to powder form.

Powder coats paints of the invention will typically be applied by electrostatic spray or fluidized bed to facilitate surface adhesion. Once the particles have adhered to the substrate, often a metal substrate, the coated article is placed into an oven to facilitate melting of the adherent powder to form a uniform coating that subsequently vulcanizes to a stable, pinhole free surface coating. The resultant powder coatings will generally be thicker than standard coatings using liquid paint mixtures comprising aqueous or organic solvents.

Because powder coatings do not rely on solvents for processing, the final coatings are essentially non-porous, which provides advantages for most antimicrobial and industrial uses contemplated herein. The activity of the surface is thereby reliant upon surface resident, surface active antimicrobial (additive) particles. Because functionalized, hydrated (organic) ion exchange resins have a pore size of approximately 1 to 2 nm (10 to 20 Å), and macroporous resins, have macropores on the order of about 20 to 100 nm (200 to 1000 Å) greater access to the embedded antimicrobial ions (e.g., Cu(II)) can be achieved. At 3.0 wt % Cu(II)-SCER loading (˜0.36 wt % Cu(II)), polyester powder coatings of the invention provide surprising antimicrobial efficacy of >3.0 log reduction against a variety of pathogenic organisms within 2.0 hours at room temperature (EPA method), while maintaining excellent mechanical properties (as gauged by scratch and impact testing). At concentrations ranging between 1.0-5.0% by weight (also tested at concentrations exceeding 6.0 wt %) mixed additive composites are also unexpectedly effective. One exemplary, mixed additive formulation (3.0 wt % Cu-SCER, 2.0 wt % Benzalkonium (BA)-SCER) demonstrated excellent mechanical properties (impact resistance) and good 2.0 hour effectiveness at room temperature using EPA methods for evaluating antibacterial efficacy. Illustrative assays in this context showed 3.6 log reductions vs. Escherichia coli and 3.2 log reductions vs. methicillin-resistant Staphylococcus aureus). For a powder loaded at 6.0 wt % Cu(II)-SCER and 2.0 wt % BA-SCER, similar reductions for these organisms were observed, 3.2 log reduction and 3.4 log reduction respectively without having any undue effect on the material's impact resistance. Applications for these coatings are numerous and include doorknobs, bed rails, wall panels, medical equipment and food processing equipment housing coatings, and a variety of touch points across clinical institutions, food processing plants, long-term care facilities, transportation hubs, and cruise ships for example.

Powder coating compositions of the invention can be employed for a variety of additional uses, including agricultural, food production and industrial, uses as contemplated herein. In one exemplary use, these compositions are effective to prevent microbial induced corrosion (MIC) when used to coat lumens of pipelines. A significant portion of “internal” pipeline corrosion results from MIC. Many organisms can contribute to corrosion as the primary cause with the main types being anaerobic sulfate-reducing bacteria (SRB), sulfur-oxidizing bacteria, iron-oxidizing and reducing bacteria, manganese-oxidizing bacteria, and acid-producing bacteria (APB). The composites used for powder coatings of pipelines may be further enhanced with other additives to minimize water and oxygen permeability for example. Exemplary embodiments include the addition of minerals to include but not limited to clay (complex silicates to include phyllosilicates, kaolinite, serpentinite, tale, vermiculite, and montmorillonite) and diatoms for example.

In an alternative embodiment of modifying surfaces with ion exchange resins, the acid group of strong or weak cation exchange resin (sulfonic acid (SCER) or carboxylic acid (WCER)) is first modified to the corresponding acid chloride and subsequently the acid chloride is reacted with a long chain (fatty) amine such as C18 (octadecyl) amine to yield a sulfonamide or amide respectively. Subsequent to this step, milling of the modified resin to small particles and incorporating of the powder into a polymer is expected to yield a material with surfaces that are more hydrophobic than the matrix polymer. The same approach can be employed using hydrophilic amines to render surfaces more hydrophilic.

### EXAMPLES

Exemplary compositions, methods, materials and devices of the invention are provided

here, which are not to be construed to limit the scope of the invention. The claims presented here, and claims contemplated to be presented in related applications or correspondence papers in this application, are supported by the entirety of the disclosure, including these examples.

All ion exchange materials for use within the invention can be purified prior to, or following association with, biologically efficacious counter-ion materials described. In certain exemplary embodiments, ion exchange materials are received from a commercial supplier and employed as received, or pre-conditioned for example by extraction with isopropyl alcohol prior to air and/or vacuum drying. All matrices such as polymer matrices used in the fabrication of the compositions such as silicone rubber, were prepared according to supplier specifications.

### Example 1

### Preparation of Strong Cation Exchange Resin-Cu(II)

Strong Cation Exchange Resin (acid form) strong cation exchange material was prepared by exposing the sodium form of IRP69, IRP69-Na in an excess of 0.1 N HCl. The material was filtered and washed with deionized water until the pH is neutral. The resin was titrated to determine the exchange capacity (EC) using established methods. The material is subsequently combined with an amount of copper (II) acetate to occupy all available sulfonic acid moieties. After a period of a couple of hours the material was filtered and washed until the eluent was free of Cu as indicated by a copper test strip (Hach). The resin was dried and milled to a particle size of 1-10-micron distribution. Elemental analysis of the compound for copper revealed copper content of 11-15 wt. % by ICP-OES.

### Example 2

### Preparation of Polystyrene Sulfonate-Cu(II)

Polystyrene sulfonic acid (Sigma Aldrich, 18 wt. % in water) was combined with an equimolar amount of copper(II) acetate in water and the reaction stirred for 2-hours until the scent of acetic acid was strong. The solution was frozen and lyophilized to yield a deep blue solid with some residual acetic acid. The material was placed under high vacuum at 80° C. to remove the acetic acid.

### Example 3

### Preparation of Polystyrene Sulfonate-Cu(II)-Modified

### Glidden Duo Premium Paint+Primer

Polystyrene sulfonate-Cu(II) was blended into acrylic latex enamel paint (Glidden Duo, Pittsburgh, PA) at a concentration of 1.5 wt. %. The fluid compositions were painted (brushed and rolled) onto a Leneta scrub test panels and the paint allowed to dry. Following extraction in deionized water, the painted samples were tested by ASTM E2180, the EPA method (2-hour), and the ISO 22196 (1-hour) assays to determine the efficacy of the Cu(II) modified acrylic latex enamel. As compared to unmodified control coated and extracted test panels, at 1.0, 1.5, and 2.5 wt. % loading of Cu(II)-SCER. The Cu(II)-SCER-modified paint demonstrated>4.3-log reductions against drug-resistant forms of methicillin-resistant Staphylococcus aureus. Table 1 illustrates activity of Cu(II)-SCER and BA-SCER modified acrylic latex enamel paint. Cu(II)-SCER=copper (II) strong cation exchange resin and BA-SCER=benzalkonium-modified strong cation exchange resin.

TABLE 1

| Assay | Active | % Active | Carrier | Log Reduction |
|---|---|---|---|---|
| EPA; | Cu(II)-PSS | 2.5 | Acrylic Latex | 5.91 |
| 2-hour | | | Enamel | |
| ISO | Cu(II)-PSS | 3.0 | Acrylic Latex | 5.44 |
| 22196; | | | Enamel | |
| 1-hour | | | | |
| ASTM | Cu(II)-PSS | 1.0 | Acrylic Latex | 4.30 |
| E2180 | | | Enamel | |
| ASTM | BA-PSS | 1.5 | Acrylic Latex | 4.37 |
| E2180 | | | Enamel | |

### Example 4

### Preparation of Polystyrene Sulfonate-Benzalkonium (BA-PSS)

Polystyrene sulfonic acid, sodium salt (Sigma Aldrich, sodium salt, molecular weight 70,000) was combined with an equimolar amount of benzalkonium chloride in water and the reaction stirred for 12-hours. The solution was frozen and lyophilized to yield a white solid. The material was placed under high vacuum at 80° C. to remove the remaining water and the resulting solid was triturated with deionized cold water to remove sodium chloride. The material product is largely insoluble in water and soluble in alcohol. The material can be precipitation purified by dissolution in alcohol and addition to water to precipitate the PSS-Benzalkonium polymer. The BA-PSS was added to acrylic latex enamel and the formulation painted onto a Leneta scrub test panel. Following extraction in deionized water the coating was evaluated against methicillin-resistant Staphylococcus aureus using the ASTM E2180 (24-hour) assay (data shown in Table 1, above).

### Example 5

### Preparation of Polystyrene Sulfonate-Benzalkonium+Polystyrene Sulfonate-Cu(II)-Modified

### Glidden Duo Premium Paint+Primer

Polystyrene sulfonate-benzalkonium and polystyrene sulfonate-Cu(II) (1.0 wt. % each) were blended into acrylic latex enamel paint (Glidden Duo). The fluid composition was painted (brushed and rolled) onto a Leneta scrub test panels and the paint allowed to dry. Following extraction in deionized water, the painted samples were tested by ASTM E2180 to determine the efficacy of the 1:1 polystyrene sulfonate-benzalkonium-polystyrene sulfonate-Cu(II) modified acrylic latex enamel architectural coating. As compared to unmodified control coated and extracted test panels, at 2.0 wt. % loading (combined) of 1:1 polystyrene sulfonate-benzalkonium+polystyrene sulfonate-Cu(II)-modified paint demonstrated>5-log reductions against drug-resistant forms of Staphylococcus aureus, Pseudomonas aeruginosa, Acinetobacter baumanii, Klebsiella pneumoniae, and Escherichia coli.

### Example 6

### Preparation of Polystyrene Sulfonate-Benzalkonium Coating Solution for Surface Disinfection

Polystyrene sulfonate-benzalkonium salt was blended into isopropyl alcohol at concentrations ranging from 0.01-5.0 wt. %. The solutions were aerosolized onto 1×1 Leneta test panel surfaces to form uniform coatings. ASTM E2180 assays were carried out against Staphylococcus aureus, Enterococcus faecalis, and Escherichia coli. The coatings were highly effective against all of the organisms yielding multi-log reductions.

In a separate coating process, spun bond non-woven (N95) respirator is coated with a 0.1 wt. % coating and the surface dried. The material remained porous, allowing air to pass.

In another coating process, a non-woven garment section was coated using the same process.

In yet another coating process, a hepa filter was coated using a 0.05 wt. % solution in isopropanol and the filter allowed to dry. The filter maintained its ability to pass air.

These coatings and coated materials are antimicrobially effective against airborne droplets comprising infectious bacterial, fungal, and viral pathogens, which efficacy is enhanced by the ability of these materials to absorb fluids, by the ability of the polyanionic polymer to interact with cell membranes, DNA or RNA, and the ability of the active ionic agent (e.g., metal ion such as Cu(II)) to disrupt biological functions (e.g., stability/function of viral RNA or DNA, cell membrane integrity and function of bacterial and fungal organisms, etc.)

### Example 7

### Fabrication of Ag-SCER Polyethylene Composite

Low density polyethylene was compounded with Ag-SCER (35.4 wt % Ag by titration) at 1.5 wt. % loading by extrusion compounding and the composition was pelletized. The material was subsequently extruded into tubing and molded into plaques. The material was evaluated using the ASTM E2180 assay against Enterococcus faecalis, Staphylococcus aureus, Pseudomonas aeruginosa, Acinelobacter baumanii, Klebsiella pneumoniae, and Escherichia coli. The data are presented in Table 2, below.

In a separate formulation, the LDPE comprised a UV stabilizer, Tosaf UV2788PE. A small amount of the resulting tubing was placed into a heated press to produce a flat sheet and the ASTM E2180 carried out. The material was tested using the ASTM E2180 against the five organisms noted and the material produced equivalent log reductions against all gram positive and gram-negative organisms. This material can be used in a coextruded format that renders only the inner lumen active. For use in an outdoor environment, the external formulation can be formulated to include a pigment to prevent darkening of the material that results with exposure to light. Thus, it can be used in the food industry to collect or dispense food products such as maple water, honey or the like.

### Example 8

### Antibacterial Activity of A Polyolefin Elastomer Modified to Include Ag-SCER Compared to A Commercial Polyolefin Elastomer Incorporating A Silver Additive Currently Used In The Food & Beverage Industry

Testing for this Example follows the protocols and details describe above for Table 1. ASTM E2180=Standard Test Method for Determining the Activity of Incorporated Antimicrobial Agent(s) In Polymeric or Hydrophobic Materials, PE=Polyethylene, POE=Polyolefin Elastomer (Engage® Dow, Midland, MI), PVC=Polyvinylchloride. PVC (Flex)=plasticized PVC, PVC (Hard)=non-plasticized PVC and Competitors Silver=Beverage Tubing (a Polyolefin Elastomer With Silver Additive). A. baumanni Acinetobacter baumanni, P. aeruginosa=Pseudomonas aeruginosa, E. faecalis=Enterococcus facalis, E. coli=Escherichia coli, K pneumoniae=Klebsiella pneumoniae, MRSA methicillin-resistant Staphylococcus aureus, S. aureus=Staphylococcus aureus.

TABLE 2

| | | | | | Log |
|---|---|---|---|---|---|
| Assay ID | Material | Active | % Load | Organism | Reduction |
| ASTM | Competitor | n/a | | Pseudomonas | 0.0 |
| E2180 | Silver | | | aeruginosa | |
| ASTM | Competitor | n/a | | Escherichia coli | 0.0 |
| E2180 | Silver | | | | |
| ASTM | Competitor | n/a | | Enterococcus faecalis | 0.0 |
| E2180 | Silver | | | | |
| ASTM | Competitor | n/a | | Methicillin-resistant | 0.0 |
| E2180 | Silver | | | Staphylococcus aureus | |
| ASTM | PE | Ag-SCER | 1.0% | Staphylococcus aureus | 4.4 |
| E2180 | | | | | |
| ASTM | PE | Ag-SCER | 1.0% | Escherichia coli | 4.3 |
| E2180 | | | | | |
| ASTM | PE | Ag-SCER | 1.0% | Pseudomonas | 4.9 |
| E2180 | | | | aeruginosa | |
| ASTM | PVC (Non- | Cu(II)- | 2.0% | Klebsiella pneumoniae | 4.5 |
| E2180 | Plasticized) | SCER | | | |
| ASTM | PVC (Non- | Cu(II)- | 2.0% | Staphylococcus aureus | 4.7 |
| E2180 | Plasticized) | SCER | | | |
| ASTM | PVC | Cu(II)- | 1.5% | Escherichia coli | 4.6 |
| E2180 | (Plasticized) | SCER | | | |
| ASTM | PVC | Cu(II)- | 1.5% | Methicillin-resistant | 4.0 |
| E2180 | (Plasticized) | SCER | | Staphylococcus aureus | |
| ASTM | PVC | Ag-SCER | 1.0% | Acinetobacter | 5.7 |
| E2180 | (Plasticized) | | | baumannii | |
| ASTM | PVC | Ag-SCER | 1.0% | Escherichia coli | 5.6 |
| E2180 | (Plasticized) | | | | |
| ASTM | PVC | Ag-SCER | 1.0% | Klebsiella pneumoniae | 6.1 |
| E2180 | (Plasticized) | | | | |
| ASTM | PVC | Ag-SCER | 1.0% | Methicillin-resistant | 5.7 |
| E2180 | (Plasticized) | | | Staphylococcus aureus | |
| ASTM | PVC | Ag-SCER | 1.0% | Pseudomonas | 7.1 |
| E2180 | (Plasticized) | | | aeruginosa | |
| ASTM | PVC | Cu(II)- | 1.5% | Methicillin-resistant | 4.2 |
| E2180 | (Plasticized) | SCER | | Staphylococcus aureus | |
| ASTM | PVC | Cu(II)- | 1.5% | Pseudomonas | 4.9 |
| E2180 | (Plasticized) | SCER | | aeruginosa | |
| ASTM | POE | Cu(II)- | 1.5% | Escherichia coli | 0.0 |
| E2180 | | SCER | | | |
| ASTM | POE | Cu(II)- | 1.5% | Methicillin-resistant | 0.0 |
| E2180 | | SCER | | Staphylococcus aureus | |
| ASTM | POE | Ag-SCER | 1.0% | Pseudomonas | 4.6 |
| E2180 | | | | aeruginosa | |
| ASTM | POE | Ag-SCER | 1.0% | Escherichia coli | 4.2 |
| E2180 | | | | | |
| ASTM | POE | Ag-SCER | 1.0% | Enterococcus faecalis | 4.1 |
| E2180 | | | | | |
| ASTM | POE | Ag-SCER | 1.0% | Methicillin-resistant | 3.8 |
| E2180 | | | | Staphylococcus aureus | |
| EPA 2.0 | POE | Ag-SCER | 1.00% | Pseudomonas | 5.0 |
| hour | | | | aeruginosa | |
| EPA 2.0 | POE | Ag-SCER | 1.00% | Enterococcus faecalis | 2.8 |
| hour | | | | | |
| ASTM | POE | Ag-SCER | 1.0% | Pseudomonas | 3.6 |
| E2180 | | | | aeruginosa | |
| ASTM | POE | Ag-SCER | 1.0% | Methicillin-resistant | 2.9 |
| E2180 | | | | Staphylococcus aureus | |
| ASTM | POE | Ag-SCER | 1.0% | Methicillin-resistant | 3.2 |
| E2180 | | | | Staphylococcus aureus | |
| ASTM | POE | Ag-SCER | 1.0% | Staphylococcus aureus | 3.6 |
| E2180 | | | | | |
| ASTM | POE | Ag-SCER | 1.0% | Pseudomonas | 3.4 |
| E2180 | | | | aeruginosa | |
| ASTM | POE | Ag-SCER | 1.0% | Staphylococcus aureus | 3.4 |
| E2180 | | | | | |
| ASTM | POE | Ag-SCER | 1.0% | Pseudomonas | 3.6 |
| E2180 | | | | aeruginosa | |
| ASTM | POE | Ag-SCER | 1.0% | Pseudomonas | 4.2 |
| E2180 | | | | aeruginosa | |
| EPA 2.0 | PE-Elastomer | Ag-SCER | 1.0% | Enterococcus faecalis | 3.0 |
| hour | Blend | | | | |
| EPA 2.0 | PE-Elastomer | Ag-SCER | 1.0% | Pseudomonas | 3.3 |
| hour | Blend | | | aeruginosa | |

### Example 9

### Acrylic Latex Enamel Paint+Primer Composition Incorporating Cu(II)-SCER, 2-Hour Room Temperature Evaluations vs. MRSA and Pseudomonas aeruginosa

SCER—Cu(II), (1-10 micron particle size, 12 wt % Cu) was blended into acrylic latex enamel paint (Glidden Duo) at a concentration of 2.5 wt. %. The fluid compositions were painted (roll coated) onto a Leneta scrub test panels and the paint allowed to dry. Following extraction in deionized water (72 hrs., 37° C.) to remove in-can preservatives to allow for the demonstration of the true efficacy of the Cu-SCER additive in the absence of the preservatives, the painted samples were tested using a modified ISO 22196 method to determine the efficacy of the Cu(II) modified acrylic latex enamel. This formulation reduced MRSA by 6.5 logs and Pseudomonas aeruginosa 7.5 logs relative to an extracted Glidden Duo control sample. Sherwin Williams Paint Shield® extracted in identical fashion provided a 2.0 log reduction against MRSA and no reduction against Pseudomonas aeruginosa and Sherwin Williams Paint Shield® not extracted provided a 5.5 log reduction against MRSA and no reduction against Pseudomonas aeruginosa. These data reveal that the efficacy of Paint Shield® is significantly impacted by aqueous washing.

### Example 10

### Glidden Duo Premium Paint+Primer Paint Composition Incorporating Benzalkonium-Strong Cation Exchange Resin+Cu(II)-Strong Cation Exchange Resin

BA-SCER and Cu(II)-SCER were blended into acrylic latex enamel paint (Glidden Duo) 1:1 at a 1.0 wt % concentration for each. The resulting composition was painted (brushed and rolled) onto a Leneta scrub test panels and the paint allowed to dry. Following extraction in deionized water, the painted samples were tested by ASTM E2180 to determine the efficacy of the Cu(II) modified acrylic latex enamel. As compared to unmodified control coated and extracted test panels, at 1.5 wt. % loading the copper-modified paint demonstrated >5-log reductions against drug-resistant forms of Staphylococcus aureus, Pseudomonas aeruginosa, Acinetobacter baumanii, Klebsiella pneumoniae, and Escherichia coli.

### Example 11

### Preparation of Polystyrene Sulfonate-Copper (II) Coating Solution for Surface Disinfection

Polystyrene sulfonate-copper salt was blended into isopropyl alcohol at concentrations ranging from 0.01-5.0 wt. %. The solutions were aerosolized onto 1×1 Leneta test panel surfaces to form uniform coatings. ASTM E2180 assays were carried out against Staphylococcus aureus, Enterococcus faecalis, and Escherichia coli. The coatings were highly effective against all of the organisms yielding multi-log reductions.

In a separate coating process, a spun bond non-woven (N95) respirator was coated with a 0.1 wt. % coating and the surface allowed to dry. The material remained porous, allowing air to pass.

In another coating process, a non-woven garment section was coated using the same process.

In yet another coating process, a hepa filter was coated using a 0.05 wt. % solution in isopropanol and the filter allowed to dry. The filter maintained its ability to pass air.

These coating, material combinations are highly effective against airborne droplets comprising infectious pathogens. The subject modified materials absorb aqueous fluid due to their hydrophilic nature and interact antimicrobially with cell membranes and or DNA or RNA of targeted pathogens. These and other surfaces coated or integrated with Cu-SCER are antimicrobially effective against enveloped viruses, such as coronavirus 229E (an accepted, predictive model subject in this context for human Covid-19 (SARS-CoV2)) at 2.0 wt % loading. At 3.0 wt % loading, these coatings and coated devices are expected to demonstrate even faster and more potent viral inactivation.

### Example 12

### 24-hour Bacterial Log Reductions Measured for 1.5 wt % Cu-SCER-Modified Semi-gloss Interior Latex Paint

ASTM E2180 data for the effectiveness of an acrylic latex enamel (ALE) semi-gloss paint incorporating 1.5 wt % Cu(II)-SCER additive (0.18 wt % Cu) coated onto a Leneta scrub test panel against 8 organisms (4 Gram positive, 4 Gram negative) are presented in Table 3, below. These data reveal that the ALE provides ≥4.3 log reductions across the diverse range of microorganisms surveyed.

TABLE 3
Cu-SCER (1.50 wt %) Loaded ALE Coating On Leneta Panel

| Organism | Log Reduction |
|---|---|
| E. Coli | 5.80 |
| S. epidermidis | 5.80 |
| S. aureus | 5.80 |
| E. faecalis | 5.80 |
| K. pneumoniae | 5.80 |
| P. aeruginosa | 5.80 |
| MRSA | 4.68 |
| A. baumanii | 4.30 |

### Example 13

### EPA Antimicrobial Effectiveness Assays for an Acrylic Latex Enamel (ALE) Semi-Gloss Paint Incorporating 1.5 wt % Cu(II)-SCER Additive Coated Onto a Leneta Scrub Test Panel

The following Example employed a published, federally-accepted assay (designed for a 2-hour analysis of antibacterial effectiveness, as provided in US EPA filing for Paint Shield® Sherwin Williams, Cleveland, OH (Efficacy Review for Sanitizer #1; EPA File Symboi67603-RE; DP Barcode: D424257), modified to include efficacy assays at 30 minutes, 1-hour and 2-hours, performed across painted surfaces with concentrations ranging from 2.5-3.0 wt % Cu(II)-SCER (0.30-0.36 wt % Cu) as well as combination formulations of BA-SCER+Cu(II)-SCER (1.0 wt % each and 1.0 wt %+2.0 wt % respectively). It is Applicant's belief that no commercial paint or coating products currently available can reduce microorganisms or viruses on surfaces by more than 3-logs within 2-hours (per a detailed survey of commercial product labels). Data from the instant studies (Table 4) demonstrate that that within an abbreviated assay period of only 30-minutes, the novel compositions of the invention effectively eradicate more than 5-logs of drug-resistant gram positive or gramnegative organisms (using comparable EPA-approved methods as published for a lead antimicrobial coating product, Paint Shield®).

TABLE 4
ASTM E2180 (24-hour), using approved EPA antibacterial evaluation
method modified to included abbreviated, 30 and 60-minute evaluations,
in addition to EPA conventional 2 hr. evaluation.

| | | | Extracted/Not | | Log |
|---|---|---|---|---|---|
| Assay ID | Active | % Load | Extracted | Organism | Reduction |
| EPA 2.0 | Cu(II)-SCER | 3.00% | Extracted | Methicillin-resistant | 7.27 |
| hour | | | | Staphylococcus | |
| | | | | aureus | |
| EPA 2.0 | Paint | n/a | Not Extracted | Methicillin-resistant | 5.71 |
| hour | Shield ® | | | Staphylococcus | |
| | | | | aureus | |
| EPA 2.0 | Paint | n/a | Extracted | Methicillin-resistant | 2.08 |
| hour | Shield ® | | | Staphylococcus | |
| | | | | aureus | |
| EPA 1.0 | Cu(II)-SCER | 3.00% | Extracted | Methicillin-resistant | 5.15 |
| hour | | | | Staphylococcus | |
| | | | | aureus | |
| EPA 1.0 | Paint | n/a | Not Extracted | Methicillin-resistant | 5.03 |
| hour | Shield ® | | | Staphylococcus | |
| | | | | aureus | |
| EPA 1.0 | Cu(II)-SCER | 3.00% | Extracted | Pseudomonas | 7.30 |
| hour | | | | aeruginosa | |
| EPA 1.0 | Paint | n/a | Not Extracted | Pseudomonas | TNTC |
| hour | Shield ® | | | aeruginosa | |
| EPA 2.0 | BA-Cu(II)- | 1.00%; 1.00% | Extracted | Methicillin-resistant | 6.04 |
| hour | SCER | | | Staphylococcus | |
| | | | | aureus | |
| EPA 1.0 | Cu(II)-SCER | 3.00% | Extracted | Acinetobacter | 6.07 |
| hour | | | | baumannii | |
| EPA 1.0 | Cu(II)-SCER | 3.00% | Extracted | Acinetobacter | 6.38 |
| hour | | | | baumannii | |
| EPA 1.0 | Paint | n/a | Not Extracted | Acinetobacter | 2.96 |
| hour | Shield ® | | | baumannii | |
| EPA 1.0 | Cu(II)-SCER | 2.50% | Extracted | Escherichia coli | 7.92 |
| hour | | | | | |
| EPA 1.0 | Zn-SCER | 2.00% | Not Extracted | Escherichia coli | TNTC |
| hour | | | | | |
| EPA 1.0 | Paint | n/a | Not Extracted | Escherichia coli | TNTC |
| hour | Shield ® | | | | |
| EPA 1.0 | Cu(II)-SCER | 2.50% | Extracted | Klebsiella | 5.30 |
| hour | | | | pneumoniae | |
| EPA 1.0 | Paint | n/a | Not Extracted | Klebsiella | TNTC |
| hour | Shield ® | | | pneumoniae | |
| EPA 30 | Cu(II)-SCER | 3.00% | Extracted | Methicillin-resistant | 2.92 |
| min | | | | Staphylococcus | |
| | | | | aureus | |
| EPA 30 | Paint | n/a | Not Extracted | Methicillin-resistant | 3.53 |
| min | Shield ® | | | Staphylococcus | |
| | | | | aureus | |
| EPA 1.0 | Cu(II)-SCER | 3.00% | Extracted | Acinetobacter | 6.07 |
| hour | | | | baumannii | |
| EPA 1.0 | Paint | n/a | Not Extracted | Acinetobacter | 2.94 |
| hour | Shield ® | | | baumannii | |
| EPA 30 | Cu(II)-SCER | 3.00% | Not Extracted | Methicillin-resistant | 5.55 |
| minute | | | | Staphylococcus | |
| | | | | aureus | |
| EPA 30 | Cu(II)-SCER | 3.00% | Extracted | Klebsiella | 5.18 |
| minute | | | | pneumoniae | |
| EPA 30 | Cu(II)-SCER | 3.00% | Not Extracted | Klebsiella | 6.06 |
| minute | | | | pneumoniae | |
| EPA 30 | BA-SCER + | 1.0%/2.0% | Not Extracted | Methicillin-resistant | 5.52 |
| minute | Cu(II)-SCER | | | Staphylococcus | |
| | | | | aureus | |
| EPA 30 | CP-SCER + | 1.0%/2.0% | Not Extracted | Methicillin-resistant | 5.08 |
| minute | Cu(II)-SCER | | | Staphylococcus | |
| | | | | aureus | |
| EPA 30 | Cu(II)-SCER | 3.00% | Not Extracted | Pseudomonas | 7.88 |
| minute | | | | aeruginosa | |
| EPA 30 | Paint | n/a | Not Extracted | Pseudomonas | 1.84 |
| minute | Shield ® | | | aeruginosa | |
| EPA 30 | Cu(II)-SCER | 3.00% | Extracted | Klebsiella | 3.65 |
| minute | | | | pneumoniae | |
| EPA 30 | Cu(II)-SCER | 3.00% | Not Extracted | Klebsiella | 6.08 |
| minute | | | | pneumoniae | |
| EPA 30 | BA-SCER + | 1.0%/2.0% | Not Extracted | Pseudomonas | 6.48 |
| minute | Cu(II)-SCER | | | aeruginosa | |
| EPA 30 | CP-SCER + | 1.0%/2.0% | Not Extracted | Pseudomonas | 5.64 |
| minute | Cu(II)-SCER | | | aeruginosa | |
| EPA 30 | Cu(II)-SCER | 3.00% | Not Extracted | Methicillin-resistant | 5.80 |
| minute | | | | Staphylococcus | |
| | | | | aureus | |

### Example 14

### Antibacterial Testing of Cu(II)-SCER-Modified Epoxy Composites and Cu(II)-SCER-Modified Acrylic Latex Enamel Following 14-Weeks of Dry Aging at 53° C.

3.0 wt % Cu(II)-SCER-modified Acrylic Latex Enamel and 3.0 wt. % Cu(II)-SCER-modified Olin (DER 331+DEH 487) Epoxy were aged for 14-weeks (53° C.) and following removal from test, the materials were evaluated for their antibacterial effectiveness. The method for testing was a modified EPA approved method for evaluating paint (30 minutes) and the existing EPA method (2.0 hours) for epoxy. Log 10 reductions that were achieved are detailed in Table 5.

TABLE 5
ASTM E2180 Log 10 Reductions (30 minute EPA method): Olin Epoxy
modified to include antimicrobial Strong Cation Exchange Resins.

| | Material/ | % | Log | Material/ | % | Log |
|---|---|---|---|---|---|---|
| Organism | Method | Active | Reduction | Method | Active | Reduction |
| Enterococcus | Latex | 3.0 | 6.7 | Olin | 3.0 | 4.0 |
| faecalis | Paint/EPA | | | Epoxy/EPA | | |
| | 30 minutes | | | 2.0 Hour | | |
| Methicillin- | Latex | 3.0 | 5.6 | Olin | 3.0 | 5.8 |
| resistant | Paint/EPA | | | Epoxy/EPA | | |
| Staphylococcus | 30 minutes | | | 2.0 Hour | | |
| aureus | | | | | | |
| Klebsiella | Latex | 3.0 | 6.1 | Olin | 3.0 | 5.1 |
| pneumoniae | Paint/EPA | | | Epoxy/EPA | | |
| | 30 minutes | | | 2.0 Hour | | |
| Acinetobacter | Latex | 3.0 | 6.4 | Olin | 3.0 | 4.5 |
| baumannii | Paint/EPA | | | Epoxy/EPA | | |
| | 30 minutes | | | 2.0 Hour | | |
| Pseudomonas | Latex | 3.0 | 7.9 | Olin | 3.0 | 6.4 |
| aeruginosa | Paint/EPA | | | Epoxy/EPA | | |
| | 30 minutes | | | 2.0 Hour | | |
| Enterobacter | Latex | 3.0 | 6.1 | Olin | 3.0 | 3.2 |
| cloacae | Paint/EPA | | | Epoxy/EPA | | |
| | 30 minutes | | | 2.0 Hour | | |
| Escherichia coli | Latex | 3.0 | 6.3 | Olin | 3.0 | 4.5 |
| | Paint/EPA | | | Epoxy/EPA | | |
| | 30 minutes | | | 2.0 Hour | | |
| Staphylococcus | Latex | 3.0 | 6.4 | Olin | 3.0 | 5.0 |
| aureus | Paint/EPA | | | Epoxy/EPA | | |
| | 30 minutes | | | 2.0 Hour | | |

*For all assays involving microbes and viruses, the calculated Log10 reductions are always dependent on the bacterial of fungal organism counts or virus counts determined from controls.

The foregoing data show significant log 10 reductions for all three of the modified epoxies tested.

### Example 15

### 24-hour Effectiveness Assay of Acrylic Latex Enamel Architectural Coating Against Candida auris by ASTM E2180

A Premium Semigloss Paint+Primer was modified to include 3.0 wt % Cu-SCER and the painted surface was evaluated against Candida auris using the ASTM E2180 assay (24-hour analysis of antifungal effectiveness). The modified paint surface was challenged with approximately 3.76×10⁴ CEUs of Candida auris. Following the 24-hour challenge, fewer than (<) 200 CFUs were collected from the modified paint surface equating a log reduction of >4.22. This exemplifies the activity of the surface against a problematic drug resistant pathogen.

### Example 16

### ISO 21702 Measurement of Antiviral Activity of a Cu(II)-SCER-Modified Premium Latex Paint+Primer Against Feline Calicivirus, an Accepted Model for Norovirus

Acrylic Latex Enamel Paint+Primer was modified at 3.0 wt. % of Cu(II)-SCER based upon the % solids as determined for a dry coating of approximately 57 wt %. The 3.0 wt. % Cu (II)-SCER-loaded paint as described was challenged with the non-enveloped feline calicivirus (FCV), a surrogate for norovirus. At 2.0 hours the viral load was reduced by >5.58 logs with no wells displaying any cytopathic effect and at 24-hours the viral load was reduced by >3.50 logs with none of the wells indicating any cytopathic effect. This indicates that within 2-hours 100% of detectable virus was inactivated.

### Example 17

### Antiviral Efficacy Evaluations of Preparation of Cu(II)-SCER-Modified Glidden Duo Premium Paint+Primer Against H1N1 Influenza Virus

Acrylic Latex Enamel Paint+Primer was modified at 3.0 wt. % of Cu(II)-SCER based upon the % solids as determined for a dry coating of approximately 57 wt %. The 3.0 wt. % Cu (II)-SCER-loaded paint as described was challenged with influenza virus (H1N1). At 2 hours, the viral load was reduced by 2.17 logs and at 24-hours by 5.08 logs.

### Example 18

### Resistance Growth of Mold on Painted Surfaces

Additional studies employed ASTM D3273-16 Standard Test Method of Resistance Growth of Mold on the Surface of Interior Coatings in an Environmental Chamber (Modified) utilizing Stachybotrys chartarum as the test organism against Leneta scrub test panels painted with PPG Glidden Premium Semigloss Paint+Primer modified to include IMS 1.5% Cu-SCER. Painted surfaces treated with Cu-SCER at 1.5% load resulted in a surface that inhibited the growth of Stachybotrys chartarum under ideal growth conditions over 4 weeks. The fungal resistance testing demonstrated that under the conditions of treatment, the 1.5 wt. % Cu-SCER loaded paint resulted in a surface that inhibited the growth of Stachybotrys chartarum under the ideal growth conditions that the test article was subjected to over a period of 4-weeks.

### Example 19

### Antibacterial Testing of Cu(II)-SCER-Modified Powder Paint Composites

Powder paints, also known as powder coatings, are solvent-less compositions of polymers that are deposited onto surfaces by electrostatic deposition or fluidized bed deposition and subsequently cured at elevated temperature to yield pinhole-free coatings (powder coatings). Table 6 provides a compilation of antibacterial testing results for a Cu (II)-SCER-Modified Polyester powder paint coating.

TABLE 6
EPA 2-hour and ASTM E2180 24-hour antibacterial data
for Cu(II)-SCER-modified polyester powder coatings.

| | | | | Log |
|---|---|---|---|---|
| Method | % Active | Organism | Inoculum | Reduction |
| ASTM E2180; | 5% Cu(II)-SCER | Methicillin-resistant | 1.00E+06 | 4.2 |
| 24-Hour | | Staphylococcus aureus | | |
| ASTM E2180 | 3% Cu(II)-SCER | Pseudomonas aeruginosa | 1.00E+06 | 4.3 |
| 24-Hour | | | | |
| ASTM E2180 | 3% Cu(II)-SCER | Methicillin-resistant | 1.00E+06 | 4.0 |
| 24 Hour | | Staphylococcus aureus | | |
| ASTM E2180 | 3% Cu(II)-SCER | Escherichia coli | 1.00E+06 | 3.7 |
| 24-Hour | | | | |
| EPA 2-hour | 3% Cu(II)-SCER | Proteus mirabilis | 1.00E+09 | 3.3 |
| EPA 2-hour | 3% Cu(II)-SCER | Enterococcus faecalis | 1.00E+09 | 5.6 |
| EPA 2-hour | 3% Cu(II)-SCER | Proteus mirabilis | 1.00E+09 | 3.6 |
| EPA 2-hour | 3% Cu(II)-SCER | Pseudomonas aeruginosa | 1.00E+09 | 6.3 |
| EPA 2-hour | Competitor | Methicillin-resistant | 1.00E+09 | 2.5 |
| | 0.5% Ag | Staphylococcus aureus | | |
| EPA 2-hour | Competitor | Pseudomonas aeruginosa | 1.00E+09 | 2.8 |
| | 0.5% Ag | | | |
| EPA 2-hour | Competitor | Proteus mirabilis | 1.00E+09 | 3.0 |
| | 0.5% Ag | | | |
| EPA 2-hour | 3% Cu(II)-SCER + | Methicillin-resistant | 1.00E+09 | 3.2 |
| | 2% BA-SCER | Staphylococcus aureus | | |
| EPA 2-hour | 3% Cu(II)-SCER + | Escherichia coli | 1.00E+09 | 3.6 |
| | 2% BA-SCER | | | |
| EPA 2-hour | 6% Cu(II)-SCER + | Escherichia coli | 1.00E+09 | 3.2 |
| | 2% BA-SCER | | | |
| EPA 2-hour | 3% Cu(II)-SCER + | Methicillin-resistant | 1.00E+09 | 3.4 |
| | 2% BA-SCER | Staphylococcus aureus | | |

Note:
Impact resistance was not altered at 8.0 wt % loading of the coating

Although the foregoing invention has been described in detail by way of example for purposes of clarity of understanding, it will be apparent to the artisan that certain changes and modifications may be practiced within the scope of the appended claims, which are presented by way of illustration not limitation. The invention will thus be understood not to be limited, except in accordance with the claims which follow or may later be presented for examination. Various publications and other references have been cited with the foregoing disclosure for economy of description. Each of these references is incorporated herein by reference in its entirety for all purposes.

### REFERENCES

Cozad, A., and R. D. Jones. 2003. Disinfection and the Prevention of Infectious Disease. Am. J. Infect. Control 31:243-254.
Aitken, C., and D. J. Jeffries. 2001. Nosocomial spread of viral disease. Clin. Microbiol. Rev. 14:528-546, Barker, J., D. Stevens, and S. F. Bloomfield. 2001
Barker, J., D. Stevens, and S. F. Bloomfield. 2001. Spread and prevention of some common viral infections in community facilities and domestic homes. J. Appl. Microbiol. 91:7-21.
England, B. L. 1982. Detection of viruses on fomites, p. 179-220. In C. P. Gerba and S. M. Goyal (ed.), Methods in environmental virology. Marcel Dekker, Inc., New York, N.Y.;
Haas, C. N., J. B. Rose, and C. P. Gerba. 1999, In Microbial agents and their transmission, p. 35-50. In C. N. Haas, J. B. Rose, and C. P. Gerba (ed.), Quantitative microbial risk assessment. J. Wiley and Sons, Inc., New York, N.Y.;
Reynolds, K. A., P. Watts, S. A. Boone, and C. P. Gerba. 2005. Occurrence of bacteria and biochemical biomarkers on public surfaces. Int. J. Environ. Health Res. 15:225-234;
Sattar, S. A. 2001. Survival of microorganisms on animate and inanimate surfaces and their disinfection, p. 195-205. In W. A. Rutala (ed.), Disinfection, sterilization and antisepsis: principles and practices in healthcare facilities. Association for Professionals in Infection Control and Epidemiology, Inc., Washington, D.C.)
Goldmann, D. A. 2000. Transmission of viral respiratory infections in the home. Pediatr. Infect. Dis. J. 19 (Suppl. 10):S97-S102.)
Standard Test Method for Determining the Activity of Incorporated Antimicrobial Agent(s) In Polymeric or Hydrophobic Materials, ASTM International, West Conshohocken, P A, 2007, Developed by Subcommittee: E35.15, Book of Standards Volume: 11.05, DOI: 10.1520/E2180-07R12, www.astm.org/

What is claimed:

1. An antimicrobial polymer composition comprising an ion exchange polymer salt material ionically associated with a cupric ion (Cu(II)), wherein the cupric ion is provided in the polymer composition at a concentration ranging from 0.0-6.0 wt %, whereby the composition effectively eliminates or reduces bacterial, fungal, and/or viral contamination of and/or transfer from a surface of the polymer composition by greater than or equal to 3.0 logs in less than or equal to 24-hours.

2. The antimicrobial polymer composition of claim 1 comprising an acrylic latex enamel water-based paint, a powder coating composite, an epoxy, or a vinyl polymer.

3. The antimicrobial polymer composition of claim 1, wherein the cupric ion is provided in the polymer composition at a concentration ranging from 1.0-3.0 wt %

4. The antimicrobial polymer composition of claim 1, wherein the cupric ion is provided in the polymer composition at a concentration ranging from 1.0-3.0 wt %, and whereby the composition effectively eliminates or reduces bacterial, fungal, and/or viral contamination of and/or transfer from a surface of the polymer composition by greater than or equal to 4.0 logs in less than or equal to 24-hours.

5. The antimicrobial polymer composition of claim 1, wherein the cupric ion is provided in the polymer composition at a concentration ranging from 1.0-3.0 wt %, and whereby the composition effectively eliminates or reduces bacterial, fungal, and/or viral contamination of and/or transfer from a surface of the polymer composition by between 4.0 and 5.0 logs in less than or equal to 24-hours.

6. The antimicrobial polymer composition of claim 1, wherein the cupric ion is provided in the polymer composition at a concentration ranging from 1.0-2.0 wt %, and whereby the composition effectively eliminates or reduces bacterial, fungal, and/or viral contamination of and/or transfer from a surface of the polymer composition by between 3.0 and 4.0 logs in less than or equal to 24-hours.

7. The antimicrobial polymer composition of claim 1, wherein the cupric ion is provided in the polymer composition at a concentration of 2.0 wt % or less, and whereby the composition effectively eliminates or reduces bacterial, fungal, and/or viral contamination of and/or transfer from a surface of the polymer composition by 3.0 logs or greater in less than or equal to 24-hours.

8. The antimicrobial polymer composition of claim 1, wherein the cupric ion is provided in the polymer composition at a concentration ranging from 1.0-3.0 wt %, and whereby the composition effectively eliminates or reduces bacterial, fungal, and/or viral contamination of and/or transfer from a surface of the polymer composition by between 4.0 and 5.0 logs in less than or equal to 2-hours.

9. The antimicrobial polymer composition of claim 1, wherein the cupric ion is provided in the polymer composition at a concentration ranging from 1.0-2.0 wt %, and whereby the composition effectively eliminates or reduces bacterial, fungal, and/or viral contamination of and/or transfer from a surface of the polymer composition by between 3.0 and 4.0 logs in less than or equal to 2-hours.

10. The antimicrobial polymer composition of claim 1, wherein the cupric ion is provided in the polymer composition at a concentration of 2.0 wt % or less, and whereby the composition effectively eliminates or reduces bacterial, fungal, and/or viral contamination of and/or transfer from a surface of the polymer composition by 3.0 logs or greater in less than or equal to 2-hours.

11. The antimicrobial polymer composition of claim 1, wherein the cupric ion is provided in the polymer composition at a concentration of 2.0 wt % or less, and wherein the composition further comprises a second biologically active agent or additive.

12. The antimicrobial polymer composition of claim 11, wherein the second biologically active agent is selected from antiseptics, antibiotics, antivirals, antifungals, oligodynamic metals, anticoagulants, anti-inflammatories, antihistamines and antioxidants.

13. The antimicrobial polymer composition of claim 11, wherein the second biologically active agent is an antiseptic.

14. The antimicrobial polymer composition of claim 13, wherein the antiseptic comprises a comprise a guanidinium group or a quaternary ammonium group.

15. The antimicrobial polymer composition of claim 13, wherein the antiseptic is selected from chlorhexidine, benzalkonium, cetylpyridinium, cetrimonium (cetrimide), polyhexamethylenebiguanide and a phosphorylcholine quaternary ammonium compound.

16. The antimicrobial polymer composition of claim 1, wherein the cupric ion is provided in the polymer composition at a concentration of 1.0-3.0 wt %, wherein the composition further comprises an antiseptic compound comprising a guanidinium group or a quaternary ammonium group, whereby the composition effectively eliminates or reduces bacterial, fungal, and/or viral contamination of and/or transfer from a surface of the polymer composition by 3.0 logs or greater in less than or equal to 8-hours.

17. The antimicrobial polymer composition of claim 16, wherein the antiseptic compound is benzalkonium.

18. The antimicrobial polymer composition of claim 1, comprising an Acrylic Latex Enamel (ALE) paint or coating composite.

19. The antimicrobial polymer composition of claim 1, comprising a powder coating composite.

20. The antimicrobial polymer composition of claim 1, which effectively eliminates or reduces viral contamination of and/or transfer from a surface of the polymer composition by a coronavirus, a norovirus, or an influenza virus by greater than or equal to 3.0 logs in less than or equal to 24-hours.

21. An antiviral polymer composition comprising an ion exchange polymer salt material ionically associated with a cupric ion (Cu(II)), wherein the cupric ion is provided in the polymer composition at a concentration ranging from 0.0-6.0 wt %, whereby the composition effectively eliminates or reduces viral contamination of and/or transfer from a surface of the polymer composition by greater than or equal to 3.0 logs in less than or equal to 24-hours.

22. The antiviral polymer composition of claim 21, which effectively eliminates or reduces viral contamination of and/or transfer from a surface of the polymer composition by a coronavirus, a norovirus, or an influenza virus by greater than or equal to 3.0 logs in less than or equal to 24-hours.

23. The antiviral polymer composition of claim 21, which effectively eliminates or reduces viral contamination of and/or transfer from a surface of the polymer composition by a coronavirus by greater than or equal to 3.0 logs in less than or equal to 24-hours.

24. The antiviral polymer composition of claim 21, which effectively eliminates or reduces viral contamination and/or transfer from a surface of the polymer composition by a norovirus to and/or by greater than or equal to 3.0 logs in less than or equal to 24-hours.

25. The antiviral polymer composition of claim 21, which effectively eliminates or reduces viral contamination of and/or transfer from a surface of the polymer composition by an influenza virus by greater than or equal to 3.0 logs in less than or equal to 24-hours.

26. The antiviral polymer composition of claim 21, which effectively eliminates or reduces viral contamination of and/or transfer from a surface of the polymer composition by a coronavirus, a norovirus, or an influenza virus by greater than or equal to 3.0 logs in less than or equal to 24-hours.

27. The antiviral polymer composition of claim 21, comprising an acrylic latex enamel water-based paint, a powder coating composite, an epoxy, or a vinyl polymer.

28. The antiviral polymer composition of claim 21, wherein the cupric ion is provided in the polymer composition at a concentration ranging from 1.0-3.0 wt %

29. The antiviral polymer composition of claim 21, wherein the cupric ion is provided in the polymer composition at a concentration ranging from 1.0-3.0 wt %, and whereby the composition effectively eliminates viral contamination of and/or transfer from a surface of the polymer composition by greater than or equal to 4.0 logs in less than or equal to 24-hours.

30. The antiviral polymer composition of claim 21, wherein the cupric ion is provided in the polymer composition at a concentration ranging from 1.0-3.0 wt %, and whereby the composition effectively eliminates or reduces viral contamination of and/or transfer from a surface of the polymer composition by between 4.0 and 5.0 logs in less than or equal to 24-hours.

31. The antiviral polymer composition of claim 21, wherein the cupric ion is provided in the polymer composition at a concentration ranging from 1.0-2.0 wt %, and whereby the composition effectively eliminates or reduces viral contamination of and/or transfer from a surface of the polymer composition by between 3.0 and 4.0 logs in less than or equal to 24-hours.

32. The antiviral polymer composition of claim 21, wherein the cupric ion is provided in the polymer composition at a concentration of 2.0 wt % or less, and whereby the composition effectively eliminates or reduces viral contamination of and/or transfer from a surface of the polymer composition by 3.0 logs or greater in less than or equal to 24-hours.

33. The antiviral polymer composition of claim 21, wherein the cupric ion is provided in the polymer composition at a concentration ranging from 1.0-3.0 wt %, and whereby the composition effectively eliminates or reduces viral contamination of and/or transfer from a surface of the polymer composition by between 4.0 and 5.0 logs in less than or equal to 2-hours.

34. The antiviral polymer composition of claim 21, wherein the cupric ion is provided in the polymer composition at a concentration ranging from 1.0-2.0 wt %, and whereby the composition effectively eliminates or reduces viral contamination of and/or transfer from a surface of the polymer composition by between 3.0 and 4.0 logs in less than or equal to 2-hours.

35. The antiviral polymer composition of claim 21, wherein the cupric ion is provided in the polymer composition at a concentration of 2.0 wt % or less, and whereby the composition effectively eliminates or reduces viral contamination of and/or transfer from a surface of the polymer composition by 3.0 logs or greater in less than or equal to 2-hours.

36. The antiviral polymer composition of claim 21, wherein the cupric ion is provided in the polymer composition at a concentration of 2.0 wt % or less, and wherein the composition further comprises a second biologically active agent or additive.

37. The antiviral polymer composition of claim 36, wherein the second biologically active agent is selected from antiseptics, antibiotics, antivirals, antifungals, oligodynamic metals, anticoagulants, anti-inflammatories, antihistamines and antioxidants.

38. The antiviral polymer composition of claim 21, comprising an Acrylic Latex Enamel (ALE) paint or coating composite.

39. The antiviral polymer composition of claim 21, comprising a powder coating composite.

40. A method for eliminating or reducing contamination of a fomite surface, and/or preventing or reducing infectious transfer of a pathogenic microorganism from a fomite surface to a susceptible human or veterinary subject, in a healthcare environment or other public facility, comprising, incorporating within or upon the fomite surface an antimicrobial composition comprising an ion exchange polymer salt material ionically associated with a cupric ion (Cu(II)), wherein the cupric ion is provided in the polymer composition at a concentration ranging from 0.0-6.0 wt %, whereby the composition effectively eliminates or reduces bacterial, fungal, and/or viral contamination of and/or infectious transfer from the fomite surface containing or coated with the polymer composition by greater than or equal to 3.0 logs in less than or equal to 24-hours.

41. The method of claim 40, wherein the polymer composition comprises an acrylic latex enamel water-based paint, a powder coating composite, an epoxy, or a vinyl polymer.

42. The method of claim 40, wherein the cupric ion is provided in the polymer composition at a concentration ranging from 1.0-3.0 wt %.

43. The method of claim 40, wherein the cupric ion is provided in the polymer composition at a concentration ranging from 1.0-3.0 wt %, and whereby the composition effectively eliminates or reduces bacterial, fungal, and/or viral contamination of and/or infectious transfer from the fomite surface containing or coated with the polymer composition by greater than or equal to 4.0 logs in less than or equal to 24-hours.

44. The method of claim 40, wherein the cupric ion is provided in the polymer composition at a concentration ranging from 1.0-3.0 wt %, and whereby the composition effectively eliminates or reduces bacterial, fungal, and/or viral contamination of and/or infectious transfer from the fomite surface containing or coated with the polymer composition by between 4.0 and 5.0 logs in less than or equal to 24-hours.

45. The method of claim 40, wherein the cupric ion is provided in the polymer composition at a concentration ranging from 1.0-2.0 wt %, and whereby the composition effectively eliminates or reduces bacterial, fungal, and/or viral contamination of and/or infectious transfer from the fomite surface containing or coated with the polymer composition by between 3.0 and 4.0 logs in less than or equal to 24-hours.

46. The method of claim 40, wherein the cupric ion is provided in the polymer composition at a concentration of 2.0 wt % or less, and whereby the composition effectively eliminates or reduces bacterial, fungal, and/or viral contamination of and/or infectious transfer from the fomite surface containing or coated with the polymer composition by 3.0 logs or greater in less than or equal to 24-hours.

47. The method of claim 40, wherein the cupric ion is provided in the polymer composition at a concentration ranging from 1.0-3.0 wt %, and whereby the composition effectively eliminates or reduces bacterial, fungal, and/or viral contamination of and/or infectious transfer from the fomite surface containing or coated with the polymer composition by between 4.0 and 5.0 logs in less than or equal to 2-hours.

48. The method of claim 40, wherein the cupric ion is provided in the polymer composition at a concentration ranging from 1.0-2.0 wt %, and whereby the composition effectively eliminates or reduces bacterial, fungal, and/or viral contamination of and/or infectious transfer from the fomite surface containing or coated with the polymer composition by between 3.0 and 4.0 logs in less than or equal to 2-hours.

49. The method of claim 40, wherein the cupric ion is provided in the polymer composition at a concentration of 2.0 wt % or less, and whereby the composition effectively eliminates or reduces bacterial, fungal, and/or viral contamination of and/or infectious transfer from the fomite surface containing or coated with the polymer composition by 3.0 logs or greater in less than or equal to 2-hours.

50. The method of claim 40, wherein the cupric ion is provided in the polymer composition at a concentration of 2.0 wt % or less, and wherein the composition further comprises a second biologically active agent or additive.

51. The method of claim 50, wherein the second biologically active agent is selected from antiseptics, antibiotics, antivirals, antifungals, oligodynamic metals, anticoagulants, anti-inflammatories, antihistamines and antioxidants.

52. The method of claim 50, wherein the second biologically active agent is an antiseptic.

53. The method of claim 52, wherein the antiseptic comprises a comprise a guanidinium group or a quaternary ammonium group.

54. The method of claim 52, wherein the antiseptic is selected from chlorhexidine, benzalkonium, cetylpyridinium, cetrimonium (cetrimide), polyhexamethylenebiguanide and a phosphorylcholine quaternary ammonium compound.

55. The method of claim 40, wherein the cupric ion is provided in the polymer composition at a concentration of 1.0-3.0 wt %, wherein the composition further comprises an antiseptic compound comprising a guanidinium group or a quaternary ammonium group, whereby the composition effectively eliminates or reduces bacterial, fungal, and/or viral contamination of and/or infectious transfer from the fomite surface containing or coated with the polymer composition by 3.0 logs or greater in less than or equal to 8-hours.

56. The method of claim 55, wherein the antiseptic compound is benzalkonium.

57. The method of claim 40, wherein the antimicrobial polymer composition comprises an Acrylic Latex Enamel (ALE) paint or coating composite.

58. The method of claim 40, wherein the antimicrobial polymer composition comprises a powder coating composite.

59. The method of claim 40, which effectively eliminates or reduces viral contamination of and/or infectious transfer from the fomite surface containing or coated with the polymer composition by a coronavirus, a norovirus, or an influenza virus by greater than or equal to 3.0 logs in less than or equal to 24-hours.

60. A wound dressing coated or invested with an antimicrobial polymer composition comprising an ion exchange polymer salt material ionically associated with a cupric ion (Cu(II)), wherein the cupric ion is provided in the polymer composition at a concentration ranging from 0.0-6.0 wt %.

61. An antimicrobial paint, lacquer, spray or powder coat composition comprising an ion exchange polymer salt material ionically associated with a cupric ion (Cu(II)), wherein the cupric ion is provided at a concentration ranging from 0.0-6.0 wt %, whereby the composition effectively eliminates or reduces bacterial, fungal, and/or viral contamination of and/or transfer from an applied surface of the paint, lacquer, spray or powder coat by greater than or equal to 3.0 logs in less than or equal to 24-hours.

62. The antimicrobial paint, lacquer, spray or powder coat composition of claim 61, comprising an acrylic latex enamel water-based paint, a powder coating composite, an epoxy, or a vinyl polymer.

63. The antimicrobial paint, lacquer, spray or powder coat composition of claim 61, wherein the cupric ion is provided at a concentration ranging from 1.0-3.0 wt %.

64. The antimicrobial paint, lacquer, spray or powder coat composition of claim 61, which is antifungally effective for agricultural use to prevent or reduce fungal infections of plants and trees.

65. The antimicrobial paint, lacquer, spray or powder coat composition of claim 61, which is antibacterially effective to reduce colonization of and/or transfer from surfaces coated by the composition by gram negative or gram-positive bacteria between 3 and 8 logs at room temperature within 1-8 hours.

66. The antimicrobial paint, lacquer, spray or powder coat composition of claim 61, which is antibacterially effective to reduce colonization of and/or transfer from surfaces coated by the composition by gram negative or gram-positive bacteria between 3-5 logs at room temperature within 1 hour or less.

67. The antimicrobial paint, lacquer, spray or powder coat composition of claim 61, which is antibacterially effective to reduce colonization of and/or transfer from surfaces coated by the composition by one or more Staphylococcus spp., methicillin-resistant Staphylococcus aureus, Enterococcus faecalis, Streptococcus spp., and Clostridium spp. bacteria between 3-5 logs at room temperature within 2-8 hours or less.

68. The antimicrobial paint, lacquer, spray or powder coat composition of claim 61, which is antibacterially effective to reduce colonization of and/or transfer from surfaces coated by the composition by Pseudomonas spp, Klebsiella spp, Proteus spp, Acinetobacter spp, Escherichia coli, and Enterobacter spp. bacteria between 3-5 logs at room temperature within 2-8 hours or less.

69. The antimicrobial paint, lacquer, spray or powder coat composition of claim 61, which is antivirally effective to reduce colonization of and/or transfer from surfaces coated by the composition by enveloped or non-enveloped viruses by 3 to 5 logs within 2-8 hours or less.

70. The antimicrobial paint, lacquer, spray or powder coat composition of claim 61, which is antivirally effective to reduce colonization of and/or transfer from surfaces coated by the composition by a norovirus spp or influenza virus spp by 3 to 5 logs within 2-8 hours or less.

71. The antimicrobial paint, lacquer, spray or powder coat composition of claim 61, which is antivirally effective against coronaviruses.

72. The antimicrobial paint, lacquer, spray or powder coat composition of claim 61, which is antivirally effective against influenza viruses.

73. The antimicrobial paint, lacquer, spray or powder coat composition of claim 61, which is antivirally effective against noroviruses.

74. The antimicrobial paint, lacquer, spray or powder coat composition of claim 61, further comprising one or more additional biologically active compound(s), agent(s) or composition(s) selected from benzalkonium polystyrene sulfonate, cetylpyridinium polystyrene sulfonate, copper polystyrene sulfonate, didecyldimethylammonium polystyrene sulfonate, zinc polystyrene sulfonate, or benzethonium or methylbenzethonium polystyrene sulfonate.
